SEQUENCE LISTING (4 sequences)

SEQ ID NO: 1 - DNA, 37 bp, Artificial
  Primer
tttcccggtacccactggacaatgccacatactttgt

SEQ ID NO: 2 - DNA, 38 bp, Artificial
  Primer
gggaaagcggccgcgcctgtgacattccgacaccgaga

SEQ ID NO: 3 - DNA, 39 bp, Artificial
  Primer
tttaaaagcttgccgccatgatgtctgcctcgcgcctgc

SEQ ID NO: 4 - DNA, 63 bp, Artificial
  Primer
aaaagcggccgctagtgatggtgatggtgatggtgatgcttattcatctgacaggtgatattc

(19) United States
(12) Patent Application Publication
(10) Pub. No.: US 2010/0015643 A1
(43) Pub. Date: Jan. 21, 2010

(54) METHOD OF QUANTITATIVE DETERMINATION OF ANTIGEN PROTEIN AND QUANTITATIVE DETERMINATION KIT THEREFOR

(75) Inventors: Chuwa Tei, Kagoshima-shi (JP); Kouji Orihara, Kagoshima-shi (JP); Kinya Nagata, Kawagoe-shi (JP); Ryutaro Oba, Kawagoe-shi (JP); Hiroyuki Hirai, Kawagoe-shi (JP)

Correspondence Address: SUGHRUE MION, PLLC, 2100 PENNSYLVANIA AVENUE, N.W., SUITE 800, WASHINGTON, DC, 20037, US

(73) Assignees: Chuwa Tei, Kagoshima-shi, Kagoshima (JP); Kouji Orihara, Kagoshima-shi, Kagoshima (JP)

(21) Appl. No.: 12/301,535

(22) PCT Filed: May 21, 2007
(86) PCT No.: PCT/JP2007/060342

§ 371 Date: Jul. 29, 2009

(30) Foreign Application Priority Data
May 19, 2006 (JP) 2006-140680
Dec. 8, 2006 (JP) 2006-332671

(51) Int. Cl. G01N 33/567

(52) U.S. Cl. 435/7.24; 435/7.21

(57) ABSTRACT

There is provided a method of quantitative determination using a flow cytometer, with which quantitative determination of cell surface protein can be effected more accurately than with current methods. The inventors have achieved the present invention by providing a method of quantitative determination of sites, per cell of a test sample, at which an antibody is bound to an antigen protein (sites/cell), characterized by preparing a calibration curve on the basis of fluorescent intensities obtained through measuring with a flow cytometer the amount of labeled antibodies against antigen protein which are bound to two or more groups of beads carrying known and different amounts of the antigen protein, and numeric values of the known amounts of the antigen protein, and further by measuring, with the flow cytometer, labeled antibodies against antigen protein after they have been reacted with test cells derived from a blood sample of a test subject, whereby digitalization is effected through comparison and conversion between the calibration curve and a fluorescence intensity obtained.

### TECHNICAL FIELD

The present invention relates to a method for quantitative determination of an antigen protein. More particularly, the present invention relates to such a quantitative determination method which makes use of a flow cytometer and to a relevant kit, whereby an antigen protein on the surface of a cell is quantitatively determined. The present invention is suitable for quantitatively determining, among other substances, toll-like receptors which are present mostly on the surface of a human leukocyte (monocyte). Quantitative determination of toll-like receptors provides a variety of indices useful in the medical field.

### BACKGROUND ART

Analysis of a substance by flow cytometry using a specific antibody for a cell surface protein is very simple in methodology itself.

For example, when leukocytes are separated from blood through density gradient centrifugation and flow cytometry is performed after a target protein for analysis is reacted with a fluorescence-labeled antibody, measurements of fluorescence intensity of the fluorescent antibody that has been bound to the cells existing within the gate of a cell fraction of interest can be obtained, whereby expression of the target protein can be investigated. This method can deal with a relatively large number of samples, and thus is widely employed to detect the presence or absence of antigens on cell surfaces and to determine percent positives. However, this method has the following limitation: although comparison is to a certain extent possible between an amount of expression and another amount of expression on the basis of the intensity level of expression by concurrently performing the measurement, if the same measurement is performed on different days, careful adjustment of measurement settings so that they are perfectly the same as those on the previous day cannot remove the difficulties encountered in studying time-course changes or in comparing the results of respective assays one another. Reasons for this include, for example, that the sensitivity of the photo-multiplier tube varies from day to day because of changes in ambient temperature, that the quality of the labeled antibodies has been degraded, and that different lots inevitably entail titer differences. Therefore, when quantification of an antigen protein present on cell surfaces is performed by use of mean fluorescence intensity (MFI), the resultant data, which are the measurements of fluorescence intensity, are not necessarily reliable and thus the measurement system cannot be accepted as sufficiently sophisticated and precise to be of use in clinical applications. Thus, it is difficult to render an accurate judgment on the infectious disease of a subject on the basis of the quantity value obtained. This issue will be described in the Examples section hereinbelow.

Heretofore, there have already been reported several common techniques for quantitatively determining TLR2 on a monocyte (i.e., TLR2 sites/cell) by use of a flow cytometer.

In one such technique (1), 4 different groups of beads to which a fluorescent substance has been attached are run on different days of measurement to thereby prepare calibration curves, and a fluorescence intensity measurement of a test sample is converted to the number of molecules of the fluorescent substance, whereby any difference in fluorescence intensity resulting from the sensitivity of the measurement device which varies from day to day can be corrected to facilitate a time-course comparison of data (QuantiBrite, the BD company). In another technique (2), 4 different groups of beads to which known quantities of mouse IgG have been attached are provided, and the beads and a test sample which has undergone a reaction with a mouse IgG primary antibody are simultaneously subjected to a secondary antibody reaction with a fluorescence-labeled anti-mouse IgG antibody, followed by measurement, whereby any difference arising in each measurement is corrected on the basis of the amount of mouse IgG, to facilitate a time-course comparison of data (QIFIKIT, the DAKO company). Both these techniques increase reliability of flow cytometry analyses to some extent, and in particular the technique (2) provides quantitative values as expressed by sites/cell. However, in technique (1), changes attributed to factors other than those related to the measurement device; e.g., degradation of antibody quality, can cause significant error (i.e., inaccuracy). Technique (2) also has the limitation that when the quality of a primary antibody which recognizes the antigen of interest has been degraded, inaccurate data which would result therefrom cannot be corrected, although correction is possible for the measurement device and secondary antibody.

### DISCLOSURE OF THE INVENTION

### Problems to be Solved by the Invention

Accordingly, an object of the present invention is to provide a quantification method employing a flow cytometer, which enables more accurate quantitative determination of a cell surface protein.

### Means to Solve the Problems

The present inventors have performed careful studies to solve the above-mentioned problems, and have found that those problems can be solved by the provision of a method of quantitative determination of the sites, per cell of a test sample, at which an antibody is bound to an antigen protein (sites/cell) (hereinafter the method may be referred to as the present quantitative method, or more simply as the present method), the method being characterized by

preparing a calibration curve on the basis of fluorescent intensities obtained through measuring with a flow cytometer the amount of labeled “antibodies against antigen protein” (hereinafter referred to as “antigen protein antibodies”) which are bound to two or more groups of beads carrying known and different amounts of the antigen protein, and numeric values of the known amounts of the antigen protein, and

measuring, with the flow cytometer, labeled antigen protein antibodies after they have been reacted with test cells derived from a blood sample of a test subject, whereby digitalization is effected through comparison and conversion between the calibration curve and a fluorescence intensity obtained.

### EFFECT OF THE INVENTION

According to the present invention, quantitative analysis of a cell surface protein can be performed with improved accuracy by means of a flow cytometer. For example, when toll-like receptors present on human monocytes are quantitatively analyzed using the method of the present invention, clinically significant effects can be obtained, which include, for example, acquisition of data useful for setting up therapeutic plans for patients suffering from infectious diseases that must be treated very cautiously.

### BRIEF DESCRIPTION OF THE DRAWINGS

FIG. 1 SDS polyacrylamide gel electrophoresis images of samples collected at different purification steps.

FIG. 2 The results of flow cytometry analysis; four groups of beads bearing different amounts of TLR2 were produced and reacted with a labeled antibody.

FIG. 3 The results of flow cytometry analysis; TLR4-bearing beads were produced and reacted with a labeled antibody.

FIG. 4 The results of flow cytometry analysis; beads bearing an antibody to CD3 were produced and reacted with a labeled antibody.

FIG. 5 Scatchard plots which were obtained through calculation of the number of moles of antibodies that were bound to the beads, the calculation being made by using the average molecular weight of IgG; i.e., 150,000, as the molecular weight of TLR2 antibody.

FIG. 6 An example of analysis obtained through use of the quantitative determination method of the present invention.

FIG. 7 A calibration curve obtained by use of the standard beads employed in the present invention.

FIG. 8 The results of a study on storage stability of TLR2 standard beads.

FIG. 9 Comparison of the number of TLR2 molecules bound to a monocyte of healthy subjects with that of patients suffering bacterial infection.

FIG. 10 The results of a comparison study on the quantitative value of TLR2 expressed on a monocyte between a group of healthy subjects and groups of patients with infections (bacterial infection, viral infection, and mycotic infection) (onset), wherein, for the patients with infection groups, quantitative determination of the number of TLR2 was performed by use of samples collected at the time of onset of the respective diseases.

FIG. 11 The results of a comparison study on the quantitative value of TLR2 expressed on a monocyte between groups of healthy subjects and patients with infection (during treatment with antibiotic administration), wherein the number of TLR2 of each patient with infection corresponds to the maximum value of data as obtained during treatment with antibiotic administration and after treatment.

FIG. 12 The results of a comparison study on the quantitative value of TLR2 expressed on a monocyte between groups of healthy subjects, patients with infectious disease (seriously ill patients with refractory pathology), and patients with viral infection (age of each subject: younger than 90 years), wherein the TLR2 number of each patient with infection corresponds to that as determined in the situation in which the clinical symptom was very severe and fatal and in which the antibiotic administered at that time was not effective.

FIG. 13 Relationship between efficacy of an antibiotic and the number of TLR2 molecules in patients with bacterial infection. The chart shows various data on the WBC count, CRP level, and the expression amount of TLR2, which are shown for two divided groups of cured and relapsed patients, wherein “marked effect” cases (of antibiotic administration) are infection cases in which reduction in WBC level to a normal range and remarkable reduction in CRP level were observed within two to three days after initiation of administration of the antibiotic, and in which rapid defervescence was observed as a clinical symptom; “weak effect” cases are infection cases in which, after initiation of administration of the antibiotic, data on any of WBC, CRP, and clinical symptoms were unstable, but in which reduction in WBC and CRP levels and improvement of the symptoms were observed after a follow-up over about one week; and “no effect” cases are those in which deterioration in WBC and CRP levels was observed in test findings and a clinical symptom was aggravated, even under administration of the antibiotic.

FIG. 14 The results of a follow-up of patients with bacterial infection over the period of the disease on the quantitative value of TLR2, wherein (a) the number of TLR2 was monitored from three weeks before to three weeks after termination of antibiotic administration, for 37 patients who had been hospitalized for bacterial infection and then gone into a remission phase through treatment with antibiotic, and the data are shown for a group of 24 patients who were completely cured with no relapse within three weeks after termination of antibiotic administration and a group of 13 patients who presented a relapse of infection within three weeks after termination of antibiotic administration, and (b) the levels of WBC and CRP and the number of TLR2 at the time of termination of administration of antibiotic were each plotted separately for the complete cure group and for the relapse group.

FIG. 15 The results of a comparison study on the quantitative value of TLR2 between groups of healthy subjects and patients infected with influenza viruses (onset), wherein the number of TLR2 corresponds to that as determined at the time of the onset of influenza infection.

FIG. 16 A graph showing the relation between the severity of common cold and the quantitative value of TLR2 on a monocyte, wherein the number of TLR2 in common cold (viral disease) cases is plotted separately for mild cases and severe cases which are divided on the basis of clinical symptoms (fever, systemic malaise, appetite, cough, runny nose, and the need for fluid replacement therapy).

FIG. 17 A graph showing the results of a follow-up of 24 patients infected with influenza viruses on the quantitative value of TLR2, wherein a typical pattern of the number of TLR2 indicating a cured state was observed in 23 patients (open circles) from the onset of influenza infection and after oral administration of an influenza-treating drug Oseltamivir, and, in one patient (solid circle), an abnormal symptom (weakness in proximal muscle) was observed during the follow-up period.

FIG. 18 The results of a comparison study on the quantitative value of TLR2 between groups of healthy subjects and patients with atrial fibrillation arrhythmia, wherein the numbers of TLR2 were plotted for the patients with atrial fibrillation arrhythmia and for the healthy subjects which are age-matched and sex-matched with the patients with atrial fibrillation arrhythmia for comparison.

FIG. 19 A graph showing relationship between the number of coronary vessels with significant stenosis and the quantitative value of TLR2, wherein patients with coronary disease were divided into three groups on the basis of the number of arterial branches with significant coronary vessel stenosis, for plotting the number of TLR2 for comparison.

### BEST MODES FOR CARRYING OUT THE INVENTION

### [Quantitative Determination Method of the Present Invention]

As the premise of the quantitative determination method of the present invention, in order to create a calibration curve, it is necessary to produce groups of beads bearing known amounts of an antigen protein. Specifically, two or more groups of beads bearing different amounts of an antigen protein (standard beads) are produced, and a quantity indicative or corresponding to the number of the antigen protein molecules on one bead of each group of beads is determined.

The two or more groups of beads refer to two or more, preferably four or more, groups of beads bearing known amounts of an antigen protein, consisting of, for example, a first group of beads bearing a certain amount of an antigen protein (i.e., ×1), a second group of beads bearing the antigen protein in an amount 10 times (i.e., ×10) that of the first group, and a third group of beads bearing the antigen protein at in amount 100 times (i.e., ×100) that of the first group. Such beads bearing different amounts of an antigen protein can be produced through changing reaction conditions, such as the amounts of antigen protein, beads, and reaction mixture.

No particular limitation is imposed on the beads employed, so long as the beads are commonly employed in the field of clinical testing. For example, latex beads or similar beads may be employed. The antigen protein may be a natural protein, but is preferably and practically a recombinant protein, which may be obtained through gene recombination.

A preferred antigen protein is a toll-like receptor protein (TLR protein). Toll-like receptor proteins will be described in detail hereinafter. Through application of the quantitative determination method of the present invention to TLR2, among TLRs, a useful indicator is provided for differentiating various infectious diseases and for monitoring disease condition. Besides this substance, TLR4 is also useful for differentiating infectious diseases, and TLR1 is a useful indicator for morbidity of a viral disease. Further, CD14, CD3, and the like, and antibodies against these (including toll-like receptor proteins) may be employed as an antigen protein to be quantitated through the method of the present invention.

An antigen protein may be bound to beads by a known method; for example, through binding a protein to commercially available amino-group-attached latex beads which have been treated with glutaraldehyde or carbodiimide; through binding to a carboxy group by use of 1-ethyl-3-(3-dimethylaminopropyl)carbodiimide; by use of a linker such as bis(sulfosuccinimidyl) suberate or disuccinimidyl suberate; through binding a protein to commercially available carboxyl-group-attached latex beads which have been treated with carbodiimide; through binding a biotin-labeled protein to commercially available streptavidin-coated latex beads; or through reaction of commercially available anti-mouse antibody-labeled magnetic beads with an anti-His antibody and then with an His-tagged antigen protein. Various methods other than those listed above may be employed.

No particular limitation is imposed on the method for determining the amount of an antigen protein carried on beads, and the amount of the antigen protein may be determined through a common method for quantitatively determining protein. For example, the number of the antigen protein molecules on a bead may be determined by providing a substance that binds specifically to the antigen protein (hereinafter referred to as an antigen-protein-binding substance), such as an antibody against the antigen protein; labeling the antigen-protein-binding substance with a labeling substance such as a radioisotope, a fluorescent dye, or a color developing dye and the corresponding non-labeled species; mixing the labeled antigen-protein-binding substance and the non-labeled antigen-protein-binding substance at different proportions; reacting each of the mixtures with the beads bearing the antigen protein and counting the amount of the labeled antigen-protein-binding substance that has bound to the antigen protein carried on the beads through such a method that is capable of detecting the selected label; creating a calibration curve by use of the counts and the mixing ratios of the labeled antigen-protein-binding substance and the non-labeled antigen-protein-binding substance (the amount of the labeled antigen-protein-binding substance that has bound to the antigen protein varies depending on the mixing ratio due to competitive reaction between the labeled antigen-protein-binding substance and the non-labeled antigen-protein-binding substance); and determining, based on the calibration curve, the number of the antigen protein molecules that have bound to a bead of each of the two or more groups of beads bearing different amounts of the antigen protein. In fact, the number determined here is not the exact number of the antigen protein molecules but is an equivalent thereto. Hereinafter, for the sake of convenience, the value determined may be referred to as the number of the antigen protein molecules.

No particular limitation is imposed on the method for storing the thus-produced beads bearing the antigen protein. Examples of the method for storing the beads include storage at a very low temperature by use of liquid nitrogen or a similar substance, storage in a lyophilized state, storage at a low temperature around −20° C., storage at a low temperature around 4° C., and storage at ambient temperature. Storage in a lyophilized state is particularly preferred, from the viewpoints of storage stability and convenience.

For quantitative determination, the two or more groups of beads bearing known and different amounts of an antigen protein may be analyzed by means of a flow cytometer by use of a fluorescence-labeled antibody against the antigen protein (the antibody may be polyclonal or monoclonal, and may be commercially available or produced through a routine method). A calibration curve is created each time measurement is performed, to determine the amount of the fluorescence-labeled antigen-protein antibody which has bound to the antigen protein on a bead of each group of beads (the amount of the labeled antibody may also be represented as the amount of the antigen, which is the counterpart of the antibody), by plotting fluorescence intensities obtained through flow cytometry against the number of the antigen protein molecules on a single bead of each group of beads. Test cells are also analyzed by means of a flow cytometer to thereby provide a fluorescence intensity corresponding to the amount of the antibody that has bound to the antigen protein on the test cells. The fluorescence intensity is converted to the number of the antigen protein molecules on each one of the test cells on the basis of the calibration curve, whereby the number of the antigen protein antibody-recognition sites per test cell (site/cell) can be numerically expressed and normalized.

For creating a calibration curve each time measurement is performed as described above, any of the following two methods may be selected. In a first method, two or more groups of beads bearing different but known amounts of an antigen protein are reacted with a fluorescence-labeled antigen-protein antibody, the two or more groups of beads are measured by means of a flow cytometer to thereby provide fluorescence intensity measurements, a calibration curve is produced through plotting the fluorescence intensity values against the amounts of the antigen protein carried, and subsequently test cells are measured for quantitative determination of the amount of the antigen protein on a test cell, whereby a calibration curve is produced independent of determination of the amount of an antigen protein expressed on test cells. In a second method, two or more groups of beads bearing known and different amounts of an antigen protein are caused to coexist with test cells, and a fluorescence-labeled antibody for antigen protein is allowed to react therewith and then analyzed, whereby production of a calibration curve and acquisition of fluorescence intensity values for determining the amount of antibodies bound to antigen protein on test cells can be achieved in an assay system of a single flow cytometer.

The quantitative determination method of the present invention is very simple and convenient, and enables use of the amount of antigen such as antigen protein as the number of specific-antibody-recognition sites on a test cell, with high sensitivity, without being affected by the elapse of time, and on a common setting basis (i.e., applicable even when the user or the flow cytometer is changed).

No particular limitation is imposed on the identity of test cell, so long as the test cell is an animal cell. The test cell may be selected in accordance with the type of the cell surface receptor to be detected. Examples of particularly preferred test cells include leukocytes (granulocytes: neutrophils, eosinophils, and basophils, and agranulocytes: lymphocytes (e.g., B cells, T cells, and NK cells), and monocytes). The test cells may be isolated from a living organism through a routine method and subjected to the quantitative determination method of the present invention.

### [Quantitative Determination Kit]

The present invention also provides a quantitative determination kit (hereinafter may be referred to as the present quantitative determination kit) for performing the aforementioned quantitative determination method of the present invention.

The present quantitative determination kit contains components which are essentially required for performing the present quantitative determination method described above and optional components which may be determined according to needed.

Specifically, the present quantitative determination kit includes, at least, two or more groups of beads bearing known and different amounts of an antigen protein. Using this kit together with a labeled antibody against the antigen protein, the aforementioned quantitative determination method of the invention may be carried out. Of course, the present quantitative determination kit may include both of the above two or more groups of beads and the antibody against the antigen protein.

The present quantitative determination kit may include, in addition to those components mentioned above, a solvent for dilution, a control antibody, wash liquid, liquid for isolating leukocytes, a reaction tube, or other components.

The quantitative determination kit of the present invention enables efficient performance of the quantitative determination method of the present invention.

### [Toll-Like Receptor]

As mentioned above, examples of preferred antigen proteins to be analyzed by the quantitative determination method of the present invention include toll-like receptors (TLRs).

Toll-like receptors (abbreviated as TLRs) was originally found in drosophilas as receptor proteins which protect them against mycotic infection. At that time, they were named “toll receptors” (Lemaitre et al., Cell, 86: 973, 1996). Thereafter, similar proteins were found in humans as homologous proteins of toll receptors (human homologues), and those proteins were then named “toll-like receptors.” Incidentally, the immune system of a living organism is generally divided into an acquired immune system, in which specificity to pathogens such as bacteria, viruses, and fungi is provided by the aid of gene rearrangement (e.g., production of an antigen-specific antibody), and a natural immune system which functions quickly by recognizing pathogens without requiring gene rearrangement. In this regards, TLRs support the natural immune system. Specifically, TLRs serve as receptors which play important roles in pattern-recognizing pathogens and inducing an initial immunoresponse and subsequently acquired immunity. Hitherto (as of the time of the present application), 12 species of TLRs have been reported (Barton and Medzhitov: Toll-like receptors and their ligands. Corr. Top. Microbial. Immunol. 2002, 270: 81-92), and TLRs (toll-like receptors) collectively refer to these receptors (TLR1 to 12). Among them, TLR2 forms a heterodimer with TLR1 or TLR6. A TLR2-TLR1 dimer recognizes a Gram-positive bacterial species as a ligand, while TLR2-TLR6 dimer recognizes a mycotic species as a ligand. TLR4 recognizes lipo-polysaccharide (endotoxin) of Gram-negative bacteria, and TLR5 recognizes flagellin, which is a protein forming flagella of bacteria. Each of TLR3, TLR7, and TLR8 recognizes double-strand RNA of a virus and single-strand RNA originating from a virus. TLR9 recognizes non-methylated CpG DNA.

### <Application of Determination of Toll-Like Receptors to Infectious Diseases>

Infectious diseases collectively refer to diseases in which a pathogen such as a bacterium, a virus, or a fungus enters a subject (host) and proliferates in the host, thereby promoting cytoclasis in the host by the pathogen (or a toxin of the pathogen) or inducing inflammatory response, whereby an organ of the host is damaged. Particularly in the treatment of compromised hosts with high fatality rates (e.g., aged people, diabetes patients, patients to whom an immunosuppressor is administered during cancer chemotherapy or after transplant of an organ, patients on a long-term steroid regimen, and patients with acquired immune deficiency syndrome), close attention and care must be taken in terms of selection, timing of change, and withdrawal of drugs. Infection is diagnosed on the basis of three essential sets of data: inflammation-related observations in a blood test (e.g., white blood cell (WBC) and C-reactive protein (CRP)), symptoms of organs (physical observations), and an identified pathogen. However, in identification of a causal bacterium, whether or not the collected specimen includes bacteria other than the causal bacterium must be taken into consideration.

In general, diagnosis of an infectious disease and identification of a causal bacterium are difficult. Particularly when a patient has no remarkable subjective symptoms or objective symptoms, an infected organ cannot be identified. Moreover, in this case, separation of a specimen and identification of a causal bacterium are impossible. Inflammation-related observations in a blood test (e.g., WBC and CRP) vary in accordance with the type of pathogen and depending on the stage of the disease. In addition, such observations have a problem that there exist a number of diseases which must be differentiated from the target disease (i.e., diseases giving blood test observations which are very similar to those of the target infectious disease). Therefore, even when blood tests are performed repeatedly, diagnosing an infectious disease is very difficult in some cases. Since pathogens causing infectious diseases include bacteria, viruses, fungi, etc., in some cases the infected pathogen is difficult to identify. Notably, antibody titer measurement employing paired sera for diagnosing viral infection is considered to provide low sensitivity. Furthermore, infectious diseases are often caused by a plurality of pathogens (mixed infectious diseases). Therefore, at present, correct diagnosis of an infectious disease is performed via a very complex procedure, making the diagnosis very difficult.

In the treatment of an infectious disease, an appropriate drug which is considered effective is selected from a variety of antibiotics, anti-viral drugs, and anti-mycotic drugs, in accordance with the type of pathogens, infected foci, host-related factors, severity, etc., and the thus-selected drug is administered to a patient in need thereof. However, as the drug administration period is prolonged, risks of severe adverse side effects such as renal function disorders, pseudomembranous enteritis, and drug-induced hepatopathy increase. In addition, thoughtless prolongation of the drug administration period increases the risk of infection of hosts, particularly infection-susceptible patients, with methicillin-resistant Staphylococcus aureus (MRSA) or a similar bacterium. Therefore, the drug administration period is preferably as short as possible. Withdrawal of drug administration in a curing stage of an infectious disease is determined from doctors' experiences and on the basis of inflammation-related observations in a blood test (e.g., WBC and CRP) and careful clinical observations. Under such circumstances, relapse of the infectious disease is difficult to avoid. Once the infectious disease has recurred, pains and burdens of relevant patients increase, with necessity of further administration of antibiotics, prolongation of the hospitalization period, admission immediately after discharge, etc.

Currently, there have never been proposed definitely effective approaches for solving the aforementioned clinical problems. The studies by the present inventors are focused on elucidation of variation features of expression amount of TLR2 among the aforementioned toll-like receptors in a cell membrane of a monocyte—a type of leucocytes—(hereinafter referred to simply as “monocyte”). According to the inventors' studies, the assay values of TLR2 can serve as clinically key indices, which are more useful for monitoring pathological conditions led by infectious diseases, as compared with conventional inflammation markers. The elucidated features of expression amount of TLR2 in monocytes are as follows. The TLR2 level does not increase in the cases of non-infectious inflammation and diseases such as ischemia, autoimmune diseases, cancer, invasion accompanying surgery, and bruise, but the TLR2 expression level of monocytes considerably increases in infectious diseases, even though inflammation is local. In addition, the degree of increase varies depending on the type of pathogens. Briefly, according to the quantitative determination method of the present invention, detection of an infectious disease and monitoring of infectious disease conditions, which include quantitative determination of a toll-like receptor protein in a blood specimen of a subject and elucidating the infection state of the host employing the determined value as an index, can be performed consistently at high sensitivity.

In other words, according to the quantitative determination method of the present invention, the TLR2 antigen amount can be provided as the number of specific monoclonal antibody recognition sites on a monocyte (surface layer), wherein the method is carried out in a simple manner and at high sensitivity, provides consistent determination values with elapse of time, and can be employed under universal conditions (employable even when the operator or the flow cytometer is changed).

In another study, mRNA of TLR2 in a monocyte is quantitatively determined instead of an antigen protein expressed on a cell membrane (Armstrong et al., Clin. Exp. Immunol. 136: 312-319, 2004), whereby clinical pathological conditions of patients with sepsis are investigated. Also in the study, attempts were made to elucidate the difference in reactivity between TLR2 and TLR4 in response to the type of causal bacteria (Gram-positive or Gram-negative). However, the change in amount of mRNA is much smaller than the change in mass of TLR2 protein on the cell membrane, which can be obtained according to the quantitative determination method of the invention. Thus, such an mRNA analysis is not thought to be evaluated as a practical clinical test. In addition, the technique for quantitating mRNA includes extraction of mRNA from cells, making the technique very complicated, and produces a large amount of waste. Thus, the technique encounters difficulty in handling a large number of clinical samples and provision of correct and consistent quantitative measurements. Hereinafter, specific indices for infectious diseases obtained through quantitative determination of TLR2 according to the method of the invention will be described.

### <Application of the Method of the Invention for Quantitatively Determining TLR2 to Various Diseases>

### (a) Differentiation of Pathogens of Infectious Diseases (Bacterial, Viral, and Mycotic)

When a value—which corresponds to the number of TLR2 recognition antibody sites per monocyte—determined through the quantitative determination method of the invention is higher than a statistical upper limit of the value of healthy subjects, the higher value can be employed as an index for determining the presence of an infectious inflammatory disease. In the case of subjects not suffering a severe bacterial infectious disease, when the value (the number of TLR2 recognition antibody sites per monocyte) determined through the quantitative determination method is higher than a statistical upper limit of the value of non-severe bacterial infectious disease subjects, the higher value can be employed as an index for determining the presence of a viral infectious disease or a mycotic infectious disease. In the case of subjects suffering an infectious disease, when the value (the number of TLR2 recognition antibody sites per monocyte) determined through the quantitative determination method falls within a statistical range of the value of healthy subjects, the value can be employed as an index for a non-infectious inflammatory disease. Examples of the non-infectious inflammatory disease include drug-induced hepatopathy, ischemic or oxygen-deficient disorders of organs, wounds including surgical invasion, collagen disease, autoimmune diseases, allergic diseases, cancers, and non-infectious hematopathy.

Speedy and correct identification of a pathogen of an infectious disease is essential for selecting an effective therapeutic drug against the infectious disease; i.e., an important step for treating the infectious disease within as short a period as possible. Particularly in the case of an infectious disease of susceptible patients, the disease is often caused by a plurality of pathogens including a bacterium, a virus, and a fungus (mixed infection). Therefore, a certain type of such diseases is very difficult to identify on the basis of variations in WBC, leucocyte fractions, and CRP, which are general inflammation-related observations in a blood test. Even though a pathogen-specific observation is obtained on the basis of graphic images such as a chest X-ray image and a chest CT image and expert knowledge and experience, speedy diagnosis (identification of a pathogen) is difficult. In this case, diagnosis is established only when certain evidence is obtained through repeatedly performing tests specific to pathogens (e.g., blood β-D-glucan level (mycotic infection) and antibody titer of paired sera (viral infection)) with diagnosis criteria. In actual clinical settings, when a disease has been completely cured through administration of a test antibiotic, the disease is identified as an infectious disease after the treatment. In some cases, possible infection with a real pathogen is not considered, while the disease is diagnosed without doubt to be the most frequently occurring bacterial infectious disease. In such a case, possibility of infection with the real pathogen is taken into account only when ineffectiveness of a certain antibiotic has been confirmed. Thereafter, other specific tests are carried out. During these further tests, an appropriate drug is not administered to the patient, and the pathogen proliferates easily. As a result, the infectious disease is aggravated.

When the quantitative determination method of the invention is employed for assaying TLR2 on monocytes, the increase profile of the TLR2 expression level varies in accordance with pathogens. Thus, mixed infection of a bacterium with a fungus or a virus, infection only with a bacterium, and a similar state can be determined in a simple manner. Furthermore, specific tests for detecting a suspected pathogen can be readily performed. Specifically, in the case of viral infection (excluding mild viral infectious diseases including ordinary cold) (Generally, an viral infectious disease exhibits symptoms specific to the causal virus upon onset thereof, and the severity of the disease is not very high so long as the disease does not progress to a complication), or in the case of an acute stage of mycotic infection (an untreated stage), the quantitative value of antibody recognition sites on a monocyte (membrane of a peripheral circulating monocyte) treated with a fluorescence-labeled anti-TLR2 antibody is as high as about 7,000 sites/cell to about 10,000 sites/cell. In the case of an acute onset stage (an untreated stage) of a bacterial infectious disease caused only by a bacterium, the quantitative value of TLR2 on monocyte membrane falls within a range of about 5,500 sites/cell to about 7,000 sites/cell. However, when the bacterial infectious disease has been aggravated over a long period of time, and virtually no antibiotics are effective, the number is as high as about 7,000 sites/cell to about 10,000 sites/cell. Based on the aforementioned features, the infection condition of a patient can be roughly evaluated through quantitative determination of TLR2 on peripheral monocyte membranes of the patient upon onset of the infectious disease. Specifically, when the TLR2 is about <7,000 sites/cell, only bacterial infection is suspected. When the TLR2 is about >7,000 sites/cell, sole-viral infection, mycotic infection, or mixed infection with a bacterium and a fungus or a virus is suspected. Notably, healthy subjects exhibit a TLR2 number of about 2,000 sites/cell to about 6,000 sites/cell. According to the previously elucidated facts, WBC virtually does not vary in viral infection and rather tends to decrease, and WBC and the neutrophil fraction increase in bacterial infection and mycotic infection. Needless to say, these conventional findings must be taken into consideration.

However, as mentioned above, the number of TLR2 antibody recognition sites per cell does not mean the absolute number of TLR2 molecules per cell. When standard beads whose particle size or TLR2 binding manner is changed are employed, or when an anti-TLR2 antibody having a different recognition site is employed, the TLR2 site count (reduced value) varies. Thus, in the present invention, the TLR2 site number (site/cell) is not limited to the aforementioned values. If required, the standard value of healthy subject and the abnormal level must be predetermined again.

### (b) Efficacy of Drugs on Infectious Diseases

The quantitative determination method is employed for quantitating TLR2 on monocytes. When the blood sample is collected from a subject to which an infectious disease therapeutic drug has been administered, and the quantitative value of TLR2 antibody recognition sites per monocyte is lowered to fall within a statistical range of measurements of the number per monocyte of healthy subjects, the infectious disease therapeutic drug is evaluated as “effective” to the disease subject. Also, when the quantitative value exceeds the statistical range of measurements of the number per monocyte of healthy subjects, the infectious disease therapeutic drug is evaluated as “not remarkably effective” to the disease subject.

In some cases, the infected organ can be immediately detected from an objective symptom or a subjective observation (pneumonia, enteritis, pyelitis, etc.). In such cases, pathogens can be identified from samples (e.g., sputum, stool, and urine) through culture of a bacterium or a fungus. The effect of an antibiotic or an antimycotic agent which is now administered or will be administered to a patient can be predicted through checking of the sensitivity of the drug to the corresponding pathogen. However, if an effective drug is selected by means of the sensitivity test and administered to a patient with an infectious disease, in some cases, virtually no efficacy of the selected drug is confirmed. One possible reason is that the aforementioned samples do not contain a causal bacterium. Also, as a result of bacteria replacement caused by drug administration, a new causal bacterium and a resistant bacterium successively appear within a short period of time. In such a case, lack of efficacy may be observed. Currently, the efficacy of a drug on a patient with an infectious disease is determined collectively on the basis of changes over time in physical observations of a patient including objective feeling, fever, and heart rate, inflammation-related observations in a blood test (e.g., WBC and CRP), etc.

The present inventors have found that measurements of TLR2 expressed on the membrane of monocytes in peripheral blood can serve as a useful index for determining the efficacy of a drug under administration. Specifically, when a blood sample collected from a subject under administration of a therapeutic drug has a TLR2 of about >7,000 sites/cell, the drug is evaluated as “practically ineffective.” In fact, the present inventors have confirmed that in the above case an increase tendency is observed in inflammation-related observations in a blood test (e.g., WBC and CRP) several days later. When a blood sample collected from such a subject has a TLR2 of about 6,000 sites/cell to about 7,000 sites/cell, a certain-level efficacy of the drug currently administered is expected. When the TLR2 is about <6,000 sites/cell, the drug is evaluated as “remarkably effective.” As compared with a conventional approach in which an antibiotic is selected on the basis of inflammation-related observations in a blood test (e.g., WBC and CRP), according to the present invention, the expression amount of TLR2 or a similar receptor is determined, and the regimen (selection and modification of drug) is determined on the basis of the quantitative value as an index. Thus, through the approach of the invention, patients do not unnecessarily suffer infection-related symptoms such as fever, and a non-effective antibiotic can be changed to an effective antibiotic without delay, whereby an infectious disease can be treated.

However, as mentioned above, the number of TLR2 antibody recognition sites per cell does not mean the absolute number of TLR2 molecules per cell. When standard beads whose particle size or TLR2 binding manner is changed are employed, or when an anti-TLR2 antibody having a different recognition site is employed, the TLR2 site count (reduced value) varies. Thus, in the present invention, the TLR2 site number (site/cell) is not limited to the aforementioned values. If required, the standard value of healthy subject and the abnormal level must be predetermined again.

### (c) Detection of Latent Infectious Disease

Since the quantitative TLR2 value sharply reflects the pathology of a long-lasting infectious disease, an infectious disease in a subclinical level (latent infection) can be detected by measuring an increase in quantitative TLR2 value. The term “latent” refers not to, for example, a virus carrier (in the case of a viral infectious disease) or a carrier (in the base of a bacterial infectious disease), but refers to a state in which proliferation of a pathogen in a host is suppressed just through the defense system of the host against the pathogen which system functions to a maximum extent, or to a latent infection state in which virtually no inflammatory response is detected in objective and subjective observations and observations in a conventional examination. However, particular cases of immuno-abnormality and immuno-resistance in relation to abnormal expression of TLR2 on monocyte membranes are excluded.

### (i) Monitoring of “Relapse” of Infectious Disease

When a blood sample is collected from a subject in an infection remission phase and during a drug rest period after administration of an infectious disease therapeutic drug, the time-lapse increase in the quantitative value of TLR2 antibody recognition sites per monocyte from the start of the drug rest period can be employed as an index for confirming relapse of infection. Furthermore, when the quantitative value of TLR2 antibody recognition sites per monocyte exceeds the sum of the statistical average of the same quantitative values of healthy subjects and double a standard deviation, the case can serve as a more reliable index for confirming relapse of infection. In the quantitative determination of TLR2 on monocytes according to the present invention, when the maximum value of the quantitative value of TLR2 antibody recognition sites per monocyte from the start of the drug rest period is lower than the statistical average of the same quantitative values of healthy subjects, the case may serve as an index for denying relapse of infection.

As described above, TLR2 analysis and monitoring of a patient with an infectious disease to whom an infectious disease therapeutic agent (e.g., an antibiotic) is administered can be appropriately performed through the quantitative determination method of the present invention. When the quantitative value of TLR2 antibody recognition sites per monocyte is lowered to a certain value or lower, administration of the infectious disease therapeutic drug is stopped, whereby relapse of an infectious disease caused by a latent pathogen can be considerably suppressed. The present inventors have proven the fact by a number of specific infectious disease cases and also statistical dependency of increase in percent relapse of the bacterial infectious disease on increase in quantitative TLR2 value. These results will be given in the Examples hereinbelow. Notably, relapse of infection must be determined on the basis of time-lapse monitoring of quantitative TLR2 value and other receptor counts and conventionally employed inflammation-related observations in a blood test (e.g., WBC and CRP). Thus, an assay of quantitative TLR2 determination alone cannot determine the timing of the start of drug rest period.

(ii) Early Detection of an Infectious Disease Before and after Treatment (e.g., Surgery) of a Non-Infectious Disease

According to the method of the invention for quantitatively determining TLR2 on monocytes, even when the blood sample is collected from a subject before or after the treatment of a non-infectious disease (e.g., surgical treatment including surgery, radiotherapy, chemotherapy, and/or physical therapy), the presence of a latent infectious disease can be reliably detected.

In the case where a patient with a non-infectious disease suffers an infectious disease in a subclinical state (latent infection) before the treatment (e.g., surgery), when the patient is considerably weakened by, for example, invasion accompanying surgery, damage by radioactive irradiation, or chemotherapy (e.g., anticancer drug), the latent infectious disease is developed due to impaired resistance to infection, possibly leading to onset of the infectious disease after surgery. In order to prevent this type of onset, performing the TLR2 quantitative determination method of the invention is recommended as a pre-surgery examination so as to check the presence of latent infection, rate of development thereof, and proliferation degree of the pathogen. Through employment of the quantitative value of TLR2 antibody recognition sites per monocyte, a latent infectious disease can be detected at high sensitivity, so long as the latent infection has lasted for a long period. Therefore, such pre-surgery detection of latent infection is more advantageous than employment of a conventional inflammation marker.

After treatment of the aforementioned non-infectious disease (note that an antibiotic is still administered after the treatment for preventing further bacterial infection even the patient is not infected at present), the levels of conventional inflammation markers (e.g., WBC and CRP) increase by invasion accompanying surgery or other factors. Therefore, when the patient suffers an infectious disease during a period of 1 to 3 weeks after surgery, use of a conventional inflammation marker encounters great difficulty in finding infection in an early stage. Even in such a case, the method of the invention for quantitatively determining TLR2 on monocytes can detect an infectious disease at very high specificity without variation which would otherwise be caused by surgical invasion, and the results obtained by the method of the invention can serve as an excellent index for monitoring post-surgery infection.

When an implantable medical apparatus/instrument such as a pacemaker, an ICD (implantable cardioverter defibrillator), or an artificial valve is implanted to a patient in surgery, great care must be taken particularly for an infectious disease (e.g., sepsis or infectious endocarditis) possibly caused by the medical apparatus/instrument. When such an infectious disease occurs, additional surgery is performed at an appropriate timing, to thereby remove the infection-causal medical apparatus/instrument, and the infection is completely treated through administration of an antibiotic. Thereafter, a new instrument of the same type is implanted to the patient through still another surgery. Note that the infectious disease cannot be completely cured by an antibiotic when the causal medical apparatus/instrument remains in the body. Such repeated surgery is a burden to the patient both from physical and economical aspects, and an enormous medical expense is imposed to the patient. The TLR2 quantitative determination method of the invention is a highly advantageous examination for determining the timing of implantation surgery of an infected patient or the timing of removal/re-implantation surgery of the patient who suffers an infectious disease caused by the implanted medical apparatus/instrument. When a patient receives a surgical operation during a strong infection stage, the risk of re-infection increases. Therefore, such strong infection is preferably suppressed to a possible maximum extent by use of a drug such as an antibiotic. The examination based on the present invention, which can detect the strength of infection, is more useful than conventional examinations, from the viewpoint of prevention of repetition of surgery.

(d) Differentiation of Infectious Diseases from Non-Infectious Diseases

As described above, in the case where the present method is used to obtain quantitative information on TRL2, if the quantitative data that represent the number of TLR2-antibody-recognition sites per monocyte is significantly higher beyond a statistically determined range of healthy subjects, such a high value may be employed as an index of an infectious inflammatory disease. Moreover, when the patient does not have a serious bacterial infection but shows a high quantitative value—that falls outside a statistically determined range for non-serious bacterial infectious disease—regarding the number of TLR2-antibody-recognition sites, such a high value may be employed as an index of a viral infectious disease or a mycotic infectious disease. Furthermore, when the patient suffers from an infectious disease and gives a quantitative value for TLR2-antibody-recognition sites which falls within a statistically determined range for healthy subjects, the normal value determined on healthy subjects may be employed as an index of a non-infectious inflammatory disease.

When a human body has inflammation (i.e., tissue destruction), it is often difficult to distinguish between the following two possible causes; i.e., a pathogen, or the presence of inflammatory disease other than infectious disease. Whatever inflammation the patient shows, early identification of the cause and setting up an appropriate therapeutic regimen is clinically very important. Nevertheless, use of conventional inflammation-related blood test data (WBC, WBC fraction, CRP, etc.) alone cannot avoid cross reactions with a broad range of non-infectious inflammation, and therefore, such conventional tests do not provide specific indices. For example, in the pathological conditions which follow herein, it is difficult to decide whether inflammation is caused by infection or not, and yet such a decision would surely be very useful in many cases. When the quantitative determination method of the present invention is applied to TLR2 existing on a monocyte, it is possible to correctly locate an infectious disease, at an early stage thereof, which onsets in combination with any of those pathological conditions. Moreover, when patients with those non-infectious inflammatory diseases are treated for complication with infectious disease, cure from the infection can be declared when the number of TLR2-antibody-recognition sites per monocyte has reached a normal level.

(i) Hepatic Disorders (Such as Viral Ones, Drug-Induced Ones, Congestive Ones, and Hypoxic Hepatitis which is Caused when the Liver is Injured by Shock)

A large population of patients suffer from liver dysfunction (including cases that are primarily identified with a liver disease and complication cases which show liver disorder), and their causes are diversified. In fact, when anomalies in liver function are observed, it is not rare that a plurality of possible factors exist, often making it difficult to identify the exact cause(s). For example, in a case where a patient with chronic hear failure developed bacterial pneumonia and was hospitalized, diagnosis may be rendered as follows: if liver dysfunction was observed during use of an antibiotic, “drug-induced liver disorder attributed to the antibiotic” or “congestive liver disorder caused by heart failure”; and if the patient's hear failure is in a serious condition, the diagnosis may be “hypoxic liver disorder resulting from cardiogenic shock.” Needless to say, viral differentiation must be performed, and in reality, although possibilities of hepatitis A, B, or C may be examined, there still remain a possible contribution by other viruses. To cope with this problem, the TLR2 quantitative determination method of the present invention can offer information which allows for differentiation acute viral liver disorder from other types of liver disorder (since an increase in the number of TLR2-antibody-recognition sites per monocyte as determined quantitatively is only observed in acute viral liver disorder cases alone, and not observed in chronic viral liver disorder or other liver disorder cases, presumably because viral propagation is extremely limited in the liver with chronic hepatitis), whereby the present method helps narrow possible causes of liver disorder.

(ii) Infections Complicated with Ischemic Organ Necrosis Such as Myocardial Infarction or Cerebral Infarction

When organ dysfunction develops from ischemia, the dysfunction itself contributes to elevate conventional inflammation-related blood test findings (WBC, WBC fraction, CRP, etc.) due to necrosis. In an acute phase of such a serious illness, bacterial infections such as pneumonia and enteritis tend to accompany at high rate. Since WBC and CRP have already risen in the acute phase of the primary pathology; i.e., ischemic organ dysfunction, conventional inflammation markers have encountered difficulty in predicting the onset of infectious disease, resulting in an unsatisfactory monitoring of infection. In such a case also, by using the present quantitative method from time to time, decision on when to start antibiotic treatment and when to change the antibiotic can be made appropriately and promptly, because the number of TLR2-antibody-recognition sites per monocyte which is quantitatively determined by use of the present quantitative method does not increase in the presence of inflammation caused by ischemic organ dysfunction but increases in the presence of infection.

(iii) Infections Complicated with Connective Tissue Disease Or Like Disease

Without any infection, patients suffering from connective tissue disease or the like disease show elevated CRP and fever in response to the activity of the disease. Therefore, when such a patient suffers from infection (not a few patients with connective tissue disease are placed under a long term low-dose steroid p.o. regimen, and such patients are in a compromised state), the patient cannot promptly become aware that something wrong (i.e., infection) has occurred in him, and as a result, diagnosis and treatment he received after visit to the hospital tend to delay. The present inventors have revealed that inflammation caused by connective tissue disease does not increase the quantitative value—the number of TLR2-antibody-recognition sites per monocyte—obtained by use of the present quantitative method. By applying the present quantitative method to TLR2 present on monocytes of a patient with connective tissue disease, diagnosis of infectious disease, which previously tended to delay, can be made precisely at an early stage, to thereby solve the problem. Also, when a patient with connective tissue disease develops infection, application of the present quantitative method to TLR2 is useful, as it helps determine as to whether the infection is completely cured during or after the treatment.

### (iv) Infectious Disease Accompanying Tumors, and Differentiation Between Tumors and Infectious Disease

When a patient has a tumor, in particular, a malignant tumor, he is predisposed to a high risk of developing an infectious disease extending over the tumor tissue, because of the destructed immune barrier. In such a case, since the malignant tumor (epithelial cancer) itself can cause fever and elevated CRP, it is difficult to decide whether infection concurs with the tumor or not. However, in principle, no increase in the number of TLR2-antibody-recognition sites per monocyte which is a quantitative value determined by the present quantitative method is caused by the cancer itself (with special cancers excluded). Therefore, the quantitative value allows for determination of the presence or absence of an infectious disease and monitoring of the infectious disease, and if infection occurs, medication against it can be administered at an appropriate timing for an appropriate period. Moreover, in setting up a treatment plan against the cancer, the present method is useful because, with sufficient control of the accompanying pathology; i.e., infection, imaging diagnosis to detect the spread of cancer can be performed relatively easily. Also, when surgical operation is selected as a treatment, by suppressing the concurring infection in advance with a medicine at a possibly maximum extent, success rate of the surgical operation, including post-surgery, can be improved. When deciding whether or not the infection complicated with tumor and present in the tumor tissue has been sufficiently suppressed, the present quantitative method directed to TLR2 provides useful information from the viewpoints of both internal medicine and surgery. In this connection, the aforementioned special cancers are cancers which, by themselves, secrete cytokine, chemokine, or other substances that are analogous to those secreted from infection.

When cancer is diagnosed, correct diagnosis is rarely made on the basis of the results of chest X-ray film or CT scanning, and generally in most cases, highly specialized techniques are required for close examination, or very expensive examination, such as PET, must be performed. For example, when a chest X-ray examination revealed an abnormal shadow which suggests a suspected lung tumor, differentiation from acute infection (e.g., cryptococcus mycotic infection) is often needed. In such a situation, a TLR2 quantitative value obtained by use of the present quantitative method facilitates to check the presence or absence of acute infectious disease, making the diagnosis of cancer fairly easy (however, for old cicatrices including old pulmonary tuberculosis and for degenerative diseases other than infectious diseases, a quantitative value representing the number of TLR2-antibody-recognition sites per monocyte, which is obtained by the present quantitative method, falls within a normal range, and therefore, differentiation of cancer from those diseases is still necessary).

(v) Infectious Diseases Complicated with Blood Diseases

In leukemia and myelodysplasia syndrome among other pathological conditions, WBC data greatly vary due to the disease's intrinsic nature and also in response to therapy (chemotherapy, marrow transplant), so that WBC cannot be used as an index of the severity of infectious disease. However, a quantitative value obtained by the present quantitative method and representing the number of TLR2-antibody-recognition sites per monocyte is consistently reproducible, even when G-CSF formulations—which are typically used for the treatment of blood diseases—are administered to increase the neutrophile count, provided that the patient does not develop any infection. Accordingly, the TLR2-directed quantitative method of the present invention is very useful for detecting complication with infectious disease and monitoring of the pathological condition of the complication.

### (e) Monitoring Complication of Grave Disease Caused by Viral Infection

Since rapid diagnosis kits for influenza became generally available in hospitals, diagnosis thereof has become easy and has allowed for selection of appropriate medications. However, there has not yet been developed an assay method which is useful as means for broadly grasping viral infections, including influenza and common cold, and objectively inferring the level of gravity thereof. Also, many viral infection cases fail to find any effective treatment method therefor. Except rare cases of grave viral infection, in most adult cases; patients are expected to recover as time passes. For this reason, hitherto, a new index which might be useful for monitoring the gravity of viral infection has not necessarily been demanded. However, the situation is completely different when the patients are newborns, infants, or infection-vulnerable patients, or when the virus species is of particular concern. In those cases, it often happens that viral infections per se develop into a serious condition, evolving into encephalomyelitis, myocarditis, liver dysfunctions, adrenalitis, testitis/oophoritis, etc. and leading to a fatally grave condition. Thus, in order to understand the pathological condition and check the efficacy of treatment, the method of the present invention for quantitatively determining TRL is very useful, as it allows for monitoring of viral infections in terms of seriousness thereof.

In the case of viral myocarditis for example (which reportedly has an incidence of about 8 out of 100000 subjects), as in the case of myocardial infarction, patients' condition often becomes fatally serious during the acute phase, so that diagnosis is difficult to differentiate it from myocardial infarction (particularly when the hospital does not have a facility for cardiac catheter angiography). To make a diagnosis of such viral myocarditis, the present method of quantitatively determining TLR2 serves as a very useful assay means, providing information on viral activity. Recent research has also revealed that there is a pathological condition, called inflammatory cardiomyopathy, in which a patient suffers from chronic inflammation in myocardial tissue after viral infection, and gradually develops dilated cardiomyopathy. In the latter case, patients have a high likelihood of having a chronic hear failure condition years ahead, and therefore, at an appropriate time, they need to be placed under a regimen of continuous oral medicine administration against chronic heart failure. Also, since inflammation occurring in the heart muscle may cause fatal arrhythmic attacks, countermeasures for preventing arrhythmia may be needed. In above-mentioned cases in which viral infection leads to persistent inflammation (of infection-related type), follow-up of the patient is important, and therefore, the present method of quantitatively determining TLR2, which detects inflammation at a satisfactory sensitivity, is a useful assay because it allows for prediction of the speed of pathological progress.

In addition, for other serious viral infections, such as encephalomyelitis and viral hepatitis, the present method of quantitatively determining TLR2 allows for an follow-up with objective viral infection indices (regarding severity and the extent of viral propagation) and monitoring from time to time, and plays a significant role in grasping the course of recovery and therapeutic effect. Quantitative data of TLR2-antibody-recognition sites per monocyte obtained by performing the quantitative method of the present invention offer important pathological information, when they are considered together with conventional inflammation-related blood profiles, assay results of blood escaped enzyme or cellular matrix components, or detection of viral antigen or virus antibody titers (when detection is possible).

Moreover, the present invention provides a further index for viral infection. That is, the present inventors have quantitated TLR1 present on a monocyte with a flow cytometer, and have found that a sub-population of patients with viral infection show elevated expression of TLR1 (note that in most bacterial or mycotic infections, this phenomenon is not observed). Thus, whether this phenomenon is present or not can serve as an significant index for making a differential diagnosis of viral infection. In this connection, the present inventors have also revealed that, in the case of viral infection, measurements of TLR1 fluorescence intensity (MFI) present a 2-peak pattern.

(f) Use as a Risk Factor for Ischemic Disease (which Allows For Prediction of Gravity of Progressive Arteriosclerosis)

To date, there have been published a vast number of reports which study causes and risk factors of arteriosclerosis from a wide variety of viewpoints such as hereditary factors, environmental factors, life styles, and gender identity (in connection with hormones). A great number of reports clearly support the fact that arteriosclerosis, as a pathological condition, involves many intricately interrelated factors which affect the progress of atherosclerotic plaque formation in the arterial wall. Thus, narrowing the research target which opens the way to prevent aggravation of arteriosclerosis appears to be difficult. Although there are certainly those referred to as “4 major risk factors” (hypertension, diabetes, hyperlipidemia, and smoking), which have long been established clinically, there remain unanswered questions in some other proposed risk factors which have been recognized for long. Under such circumstances, recent reports describe that arteriosclerosis may result from infection with chlamydia, cytomegaloviruses, or helicobacter pylori (Ramirez, et al., Ann Intern Med. 1996; 125:979-82, Saikku et al, Lancet 1988; 2:98-6, Kuo et al., J Infect Dis. 1993; 167:841-9, Melnick et al., Eur Heart J. 1999; 34:1738-43, Zhu et al., J Am Coll Cardiol. 1999; 34:1738-43, Farsak et al., J Clin Microbiol. 2000; 38:4408-11, Hoffmeister et al., Arterioscler Thromb Vasc Biol 2001; 21:427-32, Oshima et al., J Am Coll Cardiol. 2005 19; 45:1219-22). The present inventors' research results support the above findings; specifically, the inventors have found that, as compared with healthy subjects, patients with critically aggravated arteriosclerosis show a tendency of high quantitative values in terms of TLR2-antibody-recognition sites per monocyte obtained through performing the quantitative determination method of the present invention on peripheral circulating monocytes (although the increase is not as significant as in the case of infectious disease in acute phase). Thus, the method of the present invention for quantitatively determining TLR2 will make a useful assay for inferring the level of gravity of systemic arteriosclerosis. Also, given the accepted fact that signals transmitted from TLR2 to monocyte nuclei function to promote activation of monocytes, when considering the mechanism which forms the pathology of arteriosclerosis, high quantitative values in terms of TLR2-antibody-recognition sites per cell of monocyte according to the present invention may in turn be regarded an independent risk factor for aggravated arteriosclerosis. In the future, a treatment regimen directing toward reducing the quantitative value of TLR2-antibody-recognition sites for per cell of monocyte may evolve into a preventive treatment for arresting aggravation of arteriosclerosis. Therefore, the method of quantitatively determining TLR2 according to the present invention can become a useful assay means from the viewpoint of primary prevention of ischemic diseases.

### EXAMPLES

The present invention will next be described in more detail by way of examples, which should not be construed as limiting the invention thereto.

### Example 1

### Construction of TLR2 Expression Vector

TLR2 was cloned through PCR on the basis of database information by use of the following primers: F5′-tttcccggtacccactggacaatgccacatactttgt (SEQ ID NO: 1) and R5′-gggaaagcggccgcgcctgtgacattccgacaccgaga (SEQ ID NO: 2). An XbaI site was introduced upstream of a gene encoding the extracellular domain of TLR2, and six histidine tags (His tags) and an EcoRI site were introduced downstream of the gene. The template employed was prepared through the following procedure: monocytes were separated from a blood sample from a healthy volunteer who consented to the use of the sample by use of an anti-CD14 antibody labeled with magnetic beads, and RNA was extracted from the monocytes through a customary method, followed by reverse transcription of RNA by use of oligo dT or a random primer. Amplified DNA fragments were integrated into a commercially available expression vector pRC/CMV. The vector whose sequence was determined was employed as a TLR2 expression vector.

### Example 2

### Purification of TLR2 Protein

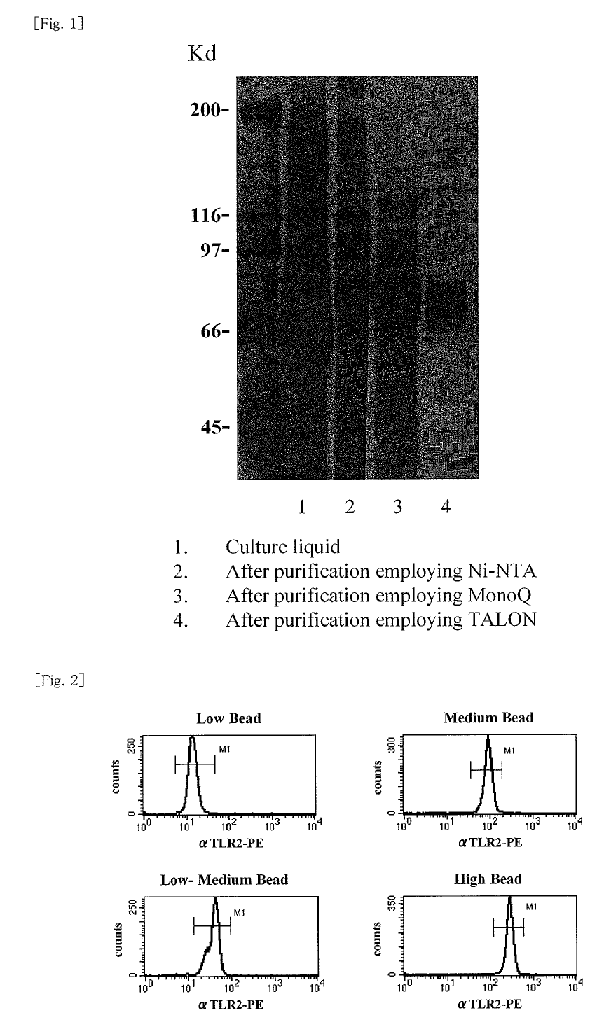

The TLR2 expression plasmid prepared in Example 1 was introduced into 293 cells through electroporation, followed by culturing in a DMEM medium containing 10% FBS in the presence of Geneticin (concentration: 0.8 mg/mL), to thereby yield cells in which the TLR2 expression plasmid is integrated into chromosomes, and which constitutively express TLR2. Subsequently, the cells were cloned by limiting dilution, and cells highly expressing TLR2 were selected. The thus-selected cells were cultured in a 293F medium under stirring for five to seven days, and the resultant culture liquid was recovered. The thus-recovered culture liquid was concentrated, and then purification was carried out by means of an Ni-NTA column having affinity for the His tags bound to recombinant TLR2, a MonoQ column (anion-exchange column), and a TALON Metal Affinity Resin column having affinity for the His tags. FIG. 1 shows the results of staining of protein with CBB after SDS polyacrylamide gel electrophoresis of samples collected at different purification stages. As shown in FIG. 1, TLR2 recombinant protein (about 76 Kd) with high purity was obtained through purification. The TLR2 recombinant protein (1.5 mg) was recovered from the culture liquid (7 L).

### Example 3

### Binding of TLR2 Protein to Beads

The TLR2 recombinant protein was bound to commercially available amino-group-surface-modified latex beads through the glutaraldehyde method. Specifically, amino-group-surface-modified polystyrene beads (diameter: 6 microns) purchased from PolyScience were washed thrice with PBS, and 8% glutaraldehyde was added to the beads, followed by inversion mixing at room temperature for one hour. After washing with PBS five times, TLR2 protein diluted with 100 mM HEPES (9.0)/PBS solution to a concentration of 3 to 0.1 mg/mL (0.1 mg/mL for Low beads, 0.6 mg/mL for Low-Medium beads, 1.6 mg/mL for Medium beads, or 1.7 mg/mL for High beads) was added to the beads, followed by inversion mixing at room temperature for two hours. Ethanolamine was added for stopping of reaction, and reaction was completed through blocking with 0.1% BSA/PBS. Through this procedure, four types of TLR2-bound beads having different TLR2 contents were prepared. Beads of each type (1×10⁵ beads) were exposed (for reaction) to 3 μg/mL PE-bound anti-TLR2 monoclonal antibody (clone name: T2.1, product of eBioscience) in 0.1% BSA/PBS, followed by analysis by means of a flow cytometer. The results are shown in FIG. 2. As shown in FIG. 2, TLR2 protein was found to bind to beads and to exhibit reactivity to the anti-TLR2 antibody. Virtually no difference was observed between these data and data obtained by use of freeze-stored TLR2-bound beads.

### Example 4

### Binding of TLR4 Protein and OKT3 Antibody to Beads

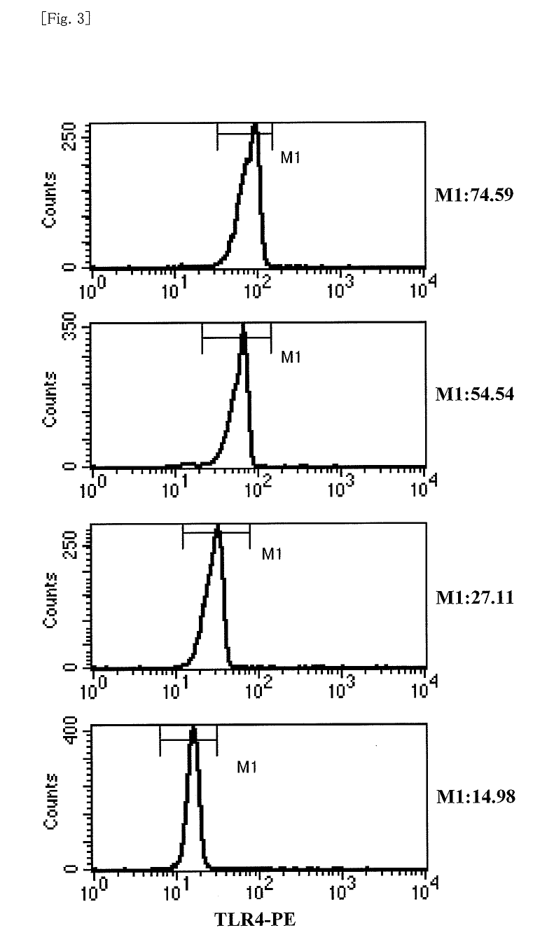

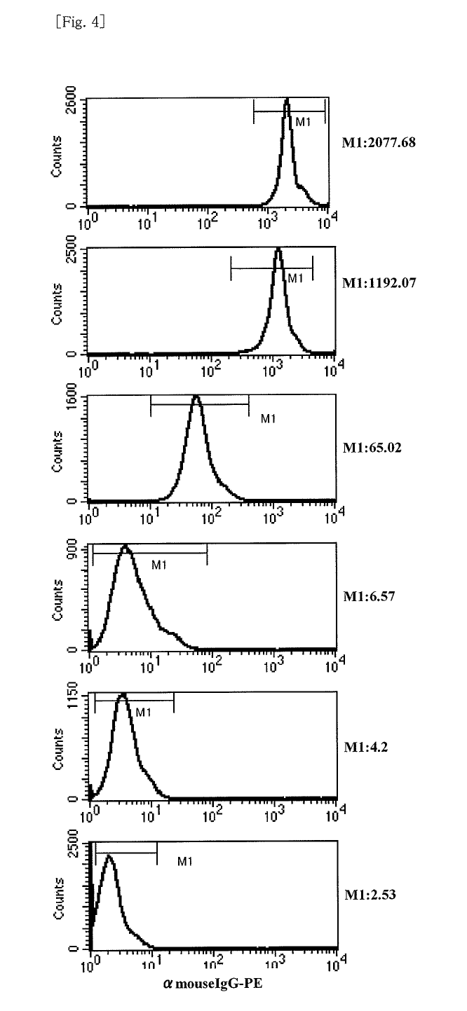

In a manner similar to that in the case of the aforementioned TLR2-bound beads, TLR4 or an antibody to CD3 (OKT3) was bound to beads, to thereby prepare standard beads (FIGS. 3 and 4). In a manner similar to the case of TLR2, TLR4 employed was prepared through cloning by use of the following primers: F5′-tttaaaagcttgccgccatgatgtctgcctcgcgcctgc (SEQ ID NO: 3) and R5′-aaaagcggccgctagtgatggtgatggtgatggtgatgcttattcatctgacaggtgatat tc (SEQ ID NO: 4) (in which an XbaI site was introduced upstream thereof and a His tag and an NotI site were introduced downstream thereof), followed by expression and protein purification. OKT3 employed was a commercially available one. Similar to the case of TLR2, these proteins were able to be used for quantitative determination.

### Example 5

### Quantitative Determination of the Number of Antibody-Recognition Sites in TLR2-Bound Standard Beads

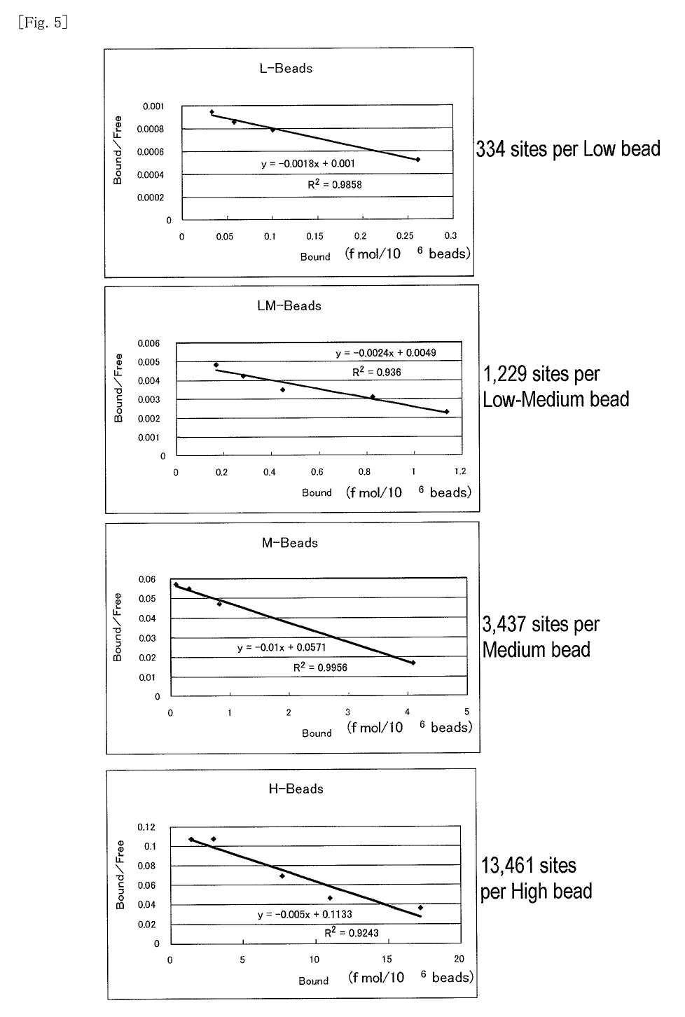

The amount of an antibody bound to TLR2-bound standard beads was determined by Scatchard plotting (i.e., the relational expression between the amounts of a bound antibody and an unbound antibody as determined after reaction). Specifically, quantitative determination was carried out as follows. Firstly, a commercially available non-labeled TLR2 antibody was labeled with ¹²⁵I through the chloramine T method. The concentration of the labeled antibody was determined through ELISA. Subsequently, in a manner similar to that described in Example 3, TLR2 protein diluted with 100 mM HEPES (9.0)/PBS solution was exposed to commercially available amino-group-surface-modified latex beads, to thereby prepare TLR2-bound beads having different TLR2 contents. Specifically, the ¹²⁵I-labeled TLR2 antibody diluted with 0.1% BSA/PBS solution to a concentration of 3 μg/mL to 6.2 ng/mL (1,500 ng/mL, 500 ng/mL, 167 ng/mL, 55.6 ng/mL, 18.5 ng/mL, or 6.2 ng/mL) was added to and reacted with the TLR2-bound latex beads (0.5×10⁶ to 5×10⁶ beads). After completion of 30-minute reaction, the beads were washed, and the amount of the antibody bound to the beads was determined by means of a gamma counter. For a control, a non-labeled TLR2 antibody (100 μg/mL) was added to and reacted with the latex beads, to thereby block the TLR2 bound to the beads, and the ¹²⁵I-labeled TLR2 antibody was added thereto, followed by determination of the amount of the ¹²⁵I-labeled antibody bound to the beads (i.e., the amount of the non-specifically bound antibody). The amount of the specifically bound antibody was determined by calculating the difference between the above-determined total amount of the antibody bound to the beads and the amount of the non-specifically bound antibody. The amount by mole of the antibody bound to the beads (bound [B]) was calculated by using the average molecular weight of IgG (i.e., 150,000) as the molecular weight of the TLR2 antibody, and the amount of the non-bound antibody (Free [F]) was determined by subtracting the amount of the bead-bound antibody from the total amount of the labeled antibody added to the reaction system. FIG. 5 shows Scatchard plots of the thus-calculated data, wherein the X-axis corresponds to [B] and the Y-axis corresponds to [B]/[F]. The value X when [F] is infinity (i.e., y=0) corresponds to the maximum amount of the antibody bound to unit bead (i.e., the number of antibody-recognition sites per bead). In the plots shown in FIG. 5, the number of antibody-recognizing (binding) sites per bead was calculated as follows: 334 sites per Low bead, 1,229 sites per Low-Medium bead, 3,437 sites per Medium bead, and 13,461 sites per High bead. The above quantitative determination was carried out a plurality of times, and the thus-obtained data were averaged. As a result, the number of antibody-binding sites in one bead of the above-prepared TLR2-bound standard beads was found to be as follow: 364 per Low bead, 1,229 per Low-Medium bead, 3,320 per Medium bead, and 14,067 per High bead. These data were employed for the below-described assay.

### Example 6

### Sample Assay Using TLR2-Bound Standard Beads

A blood sample was collected in heparin from an patient with infection who consented to the use of the sample, and a mononuclear cell fraction containing mainly lymphocytes and monocytes was separated and purified from the blood sample through density gradient centrifugation by use of Ficoll. Mononuclear cells were suspended in 0.1% BSA/PBS, and the suspension was dispensed into three tubes. A PE (phycoerythrin)-labeled anti-TLR2 antibody, a PE-labeled control (mouse IgG2a) antibody, and a PE-labeled anti-CD14 antibody (3 μg/mL each) were added to the respective three tubes, followed by reaction for 30 minutes.

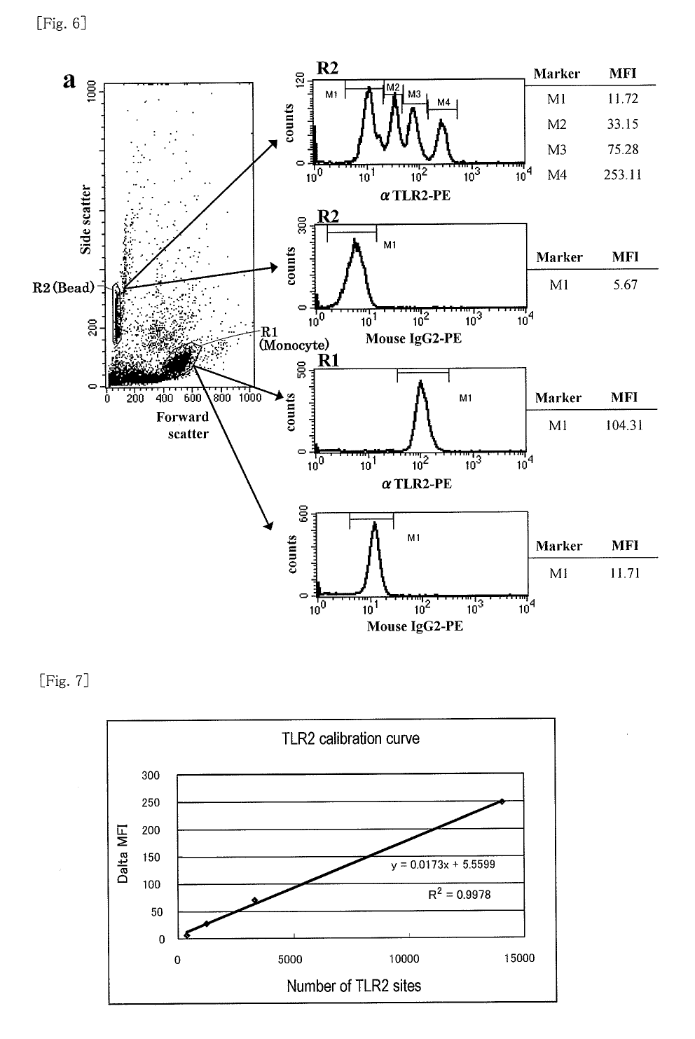

Four types of beads (Low beads to High beads) employed in Example 5 were added (1×10⁵ beads for each type) to the PE-labeled anti-TLR2 antibody and the PE-labeled control (mouse IgG2a) antibody, and these types of beads were simultaneously exposed to the antibodies for reaction. Cells and beads were washed twice with 0.1% BSA/PBS, followed by analysis by means of a flow cytometer. The results of analysis are shown in FIG. 6. CD14 is a monocyte surface marker, and a monocyte fraction (R1) can be gated on the basis of staining of the PE-labeled anti-CD14 antibody. The MFI (mean fluorescence intensity) corresponding to monocytic TLR2 and the MFI corresponding to the control antibody can be determined by use of this gate.

A bead fraction (R2) is located at a position away from the monocyte fraction and can be readily gated. In the case of staining of TLR2, four peaks are observed, and MFIs corresponding to the respective peaks can be determined. FIG. 7 shows an approximate curve obtained by plotting the thus-determined MFI data. As shown in FIG. 6, monocytic Delta MFI was 92.6 (i.e., the difference between the MFI (104.31) corresponding to the anti-TLR2 antibody and the MFI (11.71) corresponding to the control antibody). Thus, the number of TLR2-antibody-recognition sites (hereinafter may be referred to simply as “TLR2 sites”) per monocyte was determined to be 5,031 by use of the calibration curve shown in FIG. 7.

### Example 7

### Evaluation of Utility of TLR2-Bound Standard Beads in Sample Assay Under Different Conditions

The present quantitative determination method was compared with the QuantiBrite (QB) method, which is an existing method. In the QB method, a calibration curve is prepared by plotting MFI data corresponding to fluorescent-substance-bound beads having known different fluorescent substance contents; a test substance is reacted with an antibody labeled with the fluorescent substance; and the amount of the antibody bound to the test substance is determined on the basis of MFI obtained on the basis of the calibration curve, to thereby determine the amount of an antigen which is labeled (Pann, et al., Cytometry 45: 250-258, 2001).

In a manner similar to that described in Example 6, the MFI corresponding to monocytic TLR2 was measured, and the number of TLR2-antibody-recognition sites per monocyte was determined through the present quantitative determination method by use of the aforementioned beads. In addition, the amount of a fluorescent substance was determined through the QB method. A considerable variation in sensitivity in a flow cytometer upon assay on different days was simulated by changing sensitivity settings of the instrument by use of three samples (samples 1 to 3) (Table 1).

TABLE 1
Change of instrument setting

| | Sample 1 | | | Sample 2 | | | Sample 3 | | |
|---|---|---|---|---|---|---|---|---|---|
| | Delta | QB | Invention | Delta | QB | Invention | Delta | QB | Invention |
| | MFI | method | method | MFI | method | method | MFI | method | method |
| FL534 | 77.52 | 7434 | 3869 | 88.03 | 8332 | 4416 | 68.71 | 6671 | 3412 |
| FL634 | 286.43 | 7803 | 3898 | 340.43 | 9121 | 4638 | 272.67 | 7463 | 3710 |
| Average ± | 181.98 ± 57% | 7619 ± 2.4% | 3884 ± 0.37% | 214.23 ± 59% | 8727 ± 6.4% | 4527 ± 2.4% | 170.69 ± 60% | 7067 ± 5.6% | 3561 ± 4.2% |
| error | | | | | | | | | |
| (%) | | | | | | | | | |

The value determined by the QB method differs from the value determined by the present quantitative determination method, because of a difference in meaning of measurements between these methods. FL534 and FL634 correspond to sensitivity settings of the instrument. In each of these three samples, MFI is higher in the case of FL634 (i.e., higher sensitivity). In the case of the QB method or the present quantitative determination method, even when sensitivity changed, error fell within a range of ±5%. These data indicate that both the methods are useful in coping with change in sensitivity of the instrument. Subsequently, samples were changed [other three samples (samples 1 to 3)], and degradation of an antibody and error in dilution of the antibody upon assay were simulated by greatly changing the concentration of a PE-labeled anti-TLR2 antibody added. Quantitative determination was carried out in a manner similar to that described above. The results are summarized in Table 2.

TABLE 2
Change of antibody concentration

| | Sample 1 | | | Sample 2 | | | Sample 3 | | |
|---|---|---|---|---|---|---|---|---|---|
| | Delta | QB | Invention | Delta | QB | Invention | Delta | QB | Invention |
| | MFI | method | method | MFI | method | method | MFI | method | method |
| 3 μg/mL | 77.52 | 7434 | 3869 | 88.03 | 8332 | 4416 | 68.71 | 6671 | 3412 |
| 1 μg/mL | 53.68 | 5347 | 4240 | 54.15 | 5388 | 4275 | 40.4 | 4143 | 3235 |
| Average ± | 65.6 ± | 6390 ± 16% | 4055 ± 4.5% | 71.09 ± 24% | 6860 ± 21% | 4346 ± 1.6% | 54.55 ± 26% | 5407 ± 23% | 3324 ± 2.7% |
| error | 18% | | | | | | | | |
| (%) | | | | | | | | | |

As shown in Table 2, in the case of the QB method, an error of ±20% was observed, whereas in the case of the present quantitative determination method, error fell within a range of ±5% in each of the three samples. These data indicate the utility of the present quantitative determination method.

Subsequently, a test was carried out to determine the universality of the present quantitative determination method; i.e., to determine whether or not consistent quantitative data are obtained through the method by different measurers or flow cytometers. Two aliquots of the same sample were assayed by means of different measuring instruments. Errors found fell within a range of ±5%. The results are shown in Table 3.

TABLE 3
Error in the case where aliquots from the same
tube are assayed by different flow cytometers

| | Sample 1 | Sample 2 |
|---|---|---|
| Flow cytometer 1 | 5336 | 5061 |
| Flow cytometer 2 | 4983 | 5071 |
| Average ± error (%) | 5159 ± 3.4% | 5066 ± 0.09% |

Furthermore, a test was carried out to determine whether or not the same value is obtained when the same sample is assayed by means of five different flow cytometers. The results are shown in Table 4 below. As shown in Table 4, similar measurement values were obtained from the same sample, which indicates the universality of this quantitative determination system.

TABLE 4

| | Flow cytometer | | | | | | |
|---|---|---|---|---|---|---|---|
| | 1 | 2 | 3 | 4 | 5 | Average | C.V. |
| Number of | 5704 | 5144 | 5393 | 5415 | 5739 | 5479 ± 260 | 4.49 |
| TLR2 sites per | | | | | | | |
| monocyte | | | | | | | |

### Example 8

### Reproducibility on Different Days in the Quantitative Determination Method Employing TLR2-Bound Standard Beads

A test was carried out to determine whether or not similar measurement data are obtained when the same sample is assayed through the present quantitative determination method on different days. Since a blood sample cannot be freeze-stored as is, the same blood sample fails to be assayed on different days. Therefore, a blood sample was collected from a healthy volunteer who consented to the use of the sample; a mononuclear cell fraction was separated from the blood sample in a manner similar to that described in Example 6; and the mononuclear cell fraction was frozen in fetal bovine serum supplemented with 10% dimethylformamide at −80° C. and stored in liquid nitrogen for one month or more. Thereafter, the freeze-stored sample was thawed on different days and then assayed in a manner similar to that described in Example 6. The results are summarized in Table 5.

TABLE 5

| | Sample 1 | Sample 2 | Sample 3 |
|---|---|---|---|
| Day 1 | 3073 | 2081 | 2746 |
| Day 2 | 3179 | 2426 | 2670 |
| Average ± error | 3126 ± 1.7% | 2254 ± 7.7% | 2708 ± 1.4% |

As shown in Table 5, in all the experiments employing three samples, good results were obtained (i.e., error fell within a range of ±10%).

### Example 9

### Storage Stability of TLR2-Bound Standard Beads

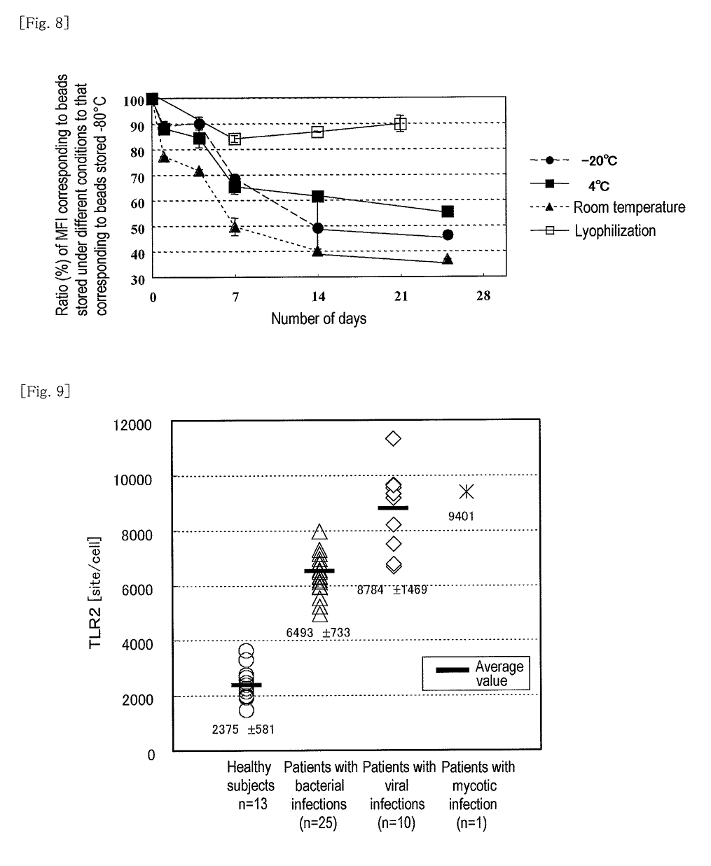

Storage conditions of the above-prepared TLR2-bound standard beads were examined. After storage in liquid nitrogen (−200° C.), the beads were stored under the following conditions (−20° C., 4° C., room temperature, and lyophilization), and assayed. The assay of the beads stored under each of the above conditions was carried out in parallel with assay of the beads stored in liquid nitrogen, and the ratio (%) of MFI corresponding to the former beads to MFI corresponding to the latter beads was determined (FIG. 8). Lyophilization of the beads was carried out in the state where the beads were suspended in 0.1% BSA/PBS supplemented with 10% sucrose. The lyophilized beads were washed once before assay. In the case of the lyophilized beads, the MFI ratio was initially reduced by about 10%, but was maintained constant thereafter. These data indicate that TLR2-bound standard beads are preferably lyophilized for storage.

Scatchard plot analysis showed that TLR2-bound standard beads stored at −80° C. are stable for at least six months.

### Example 10

### Comparison of the Present Quantitative Determination Method (Number of TLR2-Antibody-Recognition Sites Per Monocyte (Sites/Cell)) with Conventional Quantitative Determination System for TLR2 Cell Membrane Antigen (Mean Fluorescence Intensity (MFI) Value) on the Basis of Clinical Follow-Up of Subjects (Patients with Bacterial Infection)

As described above, difficulty is encountered in correctly following the clinical course of a patient over time by comparison of two or more values as determined on different days by means of a conventional flow cytometry system (MFI value), in which the conditions of a flow cytometer vary in different assays, due to change in sensitivity of the instrument, degradation of a TLR2-specific antibody, or the difference between lots. In addition, the conventional system fails to compare the values of the patient with those of a group of healthy subjects which have not been determined simultaneously with the patient values, or with numerical data obtained by a multicenter trial. The present inventors developed a quantitative determination method in which, on the basis of a prepared standard, the amount of TLR2 on monocyte membranes is represented by the number of sites recognizing a TLR2-specific monoclonal antibody, and established a method for determining the amount of TLR2 (antigen) in monocytes, to thereby achieve both consistency over time and common unit (the method is applicable to the case where assay is carried out by different measurers or flow cytometers).

In many cases, MFI value is in parallel with the number of TLR2-antibody-recognition sites per cell. In contrast, Table 6 shows four cases (patients with bacterial infection), in which MFI value is not in parallel with the number of TLR2-antibody-recognition sites per cell. In each case, follow-up was carried out to determine whether the clinical course corresponds to change in MFI value or change in numerical value as determined by the quantitative determination method developed by the present inventors. From clinical presentations (including patient's subjective symptoms, objective findings, and blood collection data (e.g., WBC count and CRP level)), a tendency toward recovery was clearly observed (cases 1 to 3), or a stable state or a slight tendency toward aggravation was observed (case 4) during the period of quantitative determination (at intervals of one to two weeks) or for several weeks thereafter. As is clear from data shown in Table 6 below, the present quantitative determination method is very suitable for understanding such a slight change in conditions of patients.

TABLE 6

| | | Blood sample | | | | | |
|---|---|---|---|---|---|---|---|
| | | collection | TLR2 | TLR2 | WBC | CRP | |
| Case | Diagnosis | date | (MFI) | (sites/cell) | (/(L) | (mg/dL) | Clinical course |
| 1 | Periodontitis | 2006. 4. 18 | 117 | 6411 | 4700 | 2.9 | Tendency toward recovery |
| | Jaw inflammation | 2006. 4. 25 | 117 | 5352 | 3300 | 0.3 | |
| 2 | Bacterial | 2006. 2. 21 | 64 | 3865 | 4700 | 1.4 | Tendency toward recovery |
| | enterocolitis | 2006. 2. 28 | 72 | 3748 | 4800 | 0.2 | |
| 3 | Bacterial | 2006. 4. 4 | 91 | 5880 | 9000 | 0.2 | Tendency toward recovery |
| | pneumonia | 2006. 4. 18 | 93 | 5074 | 8800 | 0.1 | |
| 4 | Bacterial | 2006. 3. 28 | 86 | 4703 | 3900 | 0.1 | Stable, or slight tendency |
| | pneumonia | 2006. 4. 4 | 75 | 4871 | 5200 | 0.1 | toward aggravation |

### Example 11-1

### Assay of TLR2 Expression in Samples from Healthy Subjects and Patients with Infection by Use of TLR2-Bound Standard Beads

Blood was collected from 13 healthy volunteers and 36 patients with infections (25 for bacterial infection, 10 for viral infection, and 1 for mycotic infection) who consented to the use of their blood, and the number of TLR2 sites per monocyte was determined in a manner similar to that described in Example 5. As a result, as shown in FIG. 9, in the group of the healthy volunteers, the average number of TLR2 sites per monocyte was 2,370±581, whereas in the group of the patients with bacterial infection, the average number of TLR2 sites per monocyte was 6,493±733, and in the group of the patients with viral infection, the average number of TLR2 sites per monocyte was 8,784±1,469 (i.e., the number of TLR2 sites per monocyte in the groups of patients with infection was significantly greater than that in the healthy volunteer group). The number of TLR2 sites per monocyte in the group of the patients with viral infection was greater than that in the group of the patients with bacterial infection.

### Example 11-2

### Differentiation of Types of Pathogens (Bacterium, Virus, and Fungus) of Infection

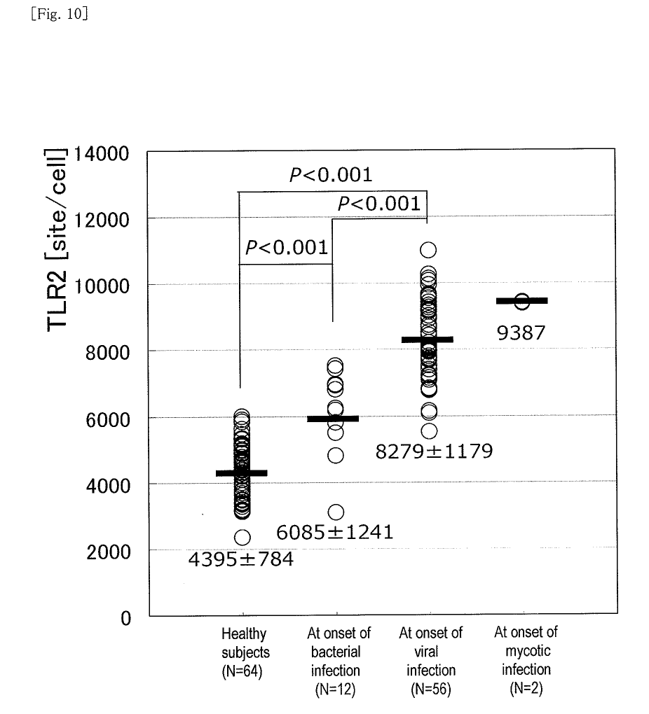

FIG. 10 shows data on the quantitative value determined for TLR2 on a monocyte (hereinafter may be referred to simply as “TLR2 count”) as determined at the time of onset (i.e., at the time of consultation in hospital immediately after development of subjective symptoms) of infections (bacterial infection, viral infection, and mycotic infection). In bacterial infection cases (at the time of no antibiotic administration), the level of TLR2 expression tended to increase. However, in many bacterial infection cases, TLR2 count fell within the normal range. In contrast, in viral infection cases, TLR2 count considerably exceeded the normal range and reached a very high level at the time of development of subjective symptoms. In two mycotic infection cases, there was a tendency similar to that in the viral infection cases. At the time of onset of the diseases, there were some cases showing typical symptoms for clearly differentiating, by a conventional inflammatory marker, viral infection from bacterial infection or mycotic infection (in general, in many bacterial or mycotic infection cases, an increase in WBC count is observed, followed by observation of an increase in CRP level, whereas in almost all viral infection cases (other than adenovirus infection), an increase in WBC count and a considerable increase in CRP level are not observed). However, in some cases, WBC count and CRP level fell within the respective normal ranges, and even an infectious state was not shown. Also, in some viral infection cases, there was a possibility of bacterial infection. In full comprehension of the aforementioned feature of an increase in level of TLR2 expression on a monocyte, studies on the number of TLR2 sites on a peripheral blood monocyte from patients with infection—as determined by the present quantitative determination method immediately after onset of the disease—provided some guideline for, for example, the following questions “Is there no doubt about, for example, bacterial infection or connective tissue disease (which will be described hereinbelow) in consideration of viral cold?,” “Can attention be focused only on bacterial infection?,” and “Is there no possibility of mixed infection?”, and also provided information as an aid for diagnosis at an early stage of the disease. It was shown that examination of the level of TLR2 expression on a monocyte (in addition to examination of WBC count, leukocyte fraction, and CRP level) at the onset of the disease leads to reliable diagnosis based on objective data.

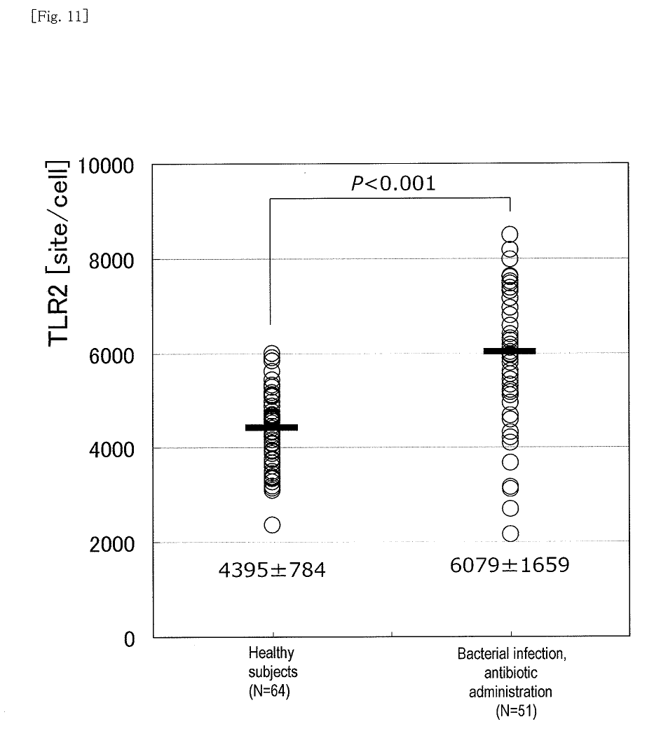

FIG. 11 shows comparison in TLR2 count as determined through the present quantitative determination method between healthy subjects and patients with bacterial infection, wherein the TLR2 count of each patient corresponds to the maximum value of data as obtained during treatment with antibiotic administration and in a remission phase.

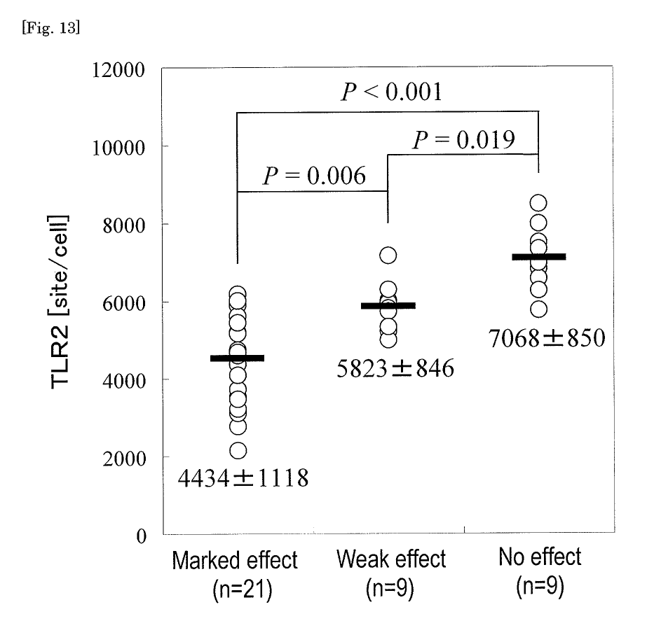

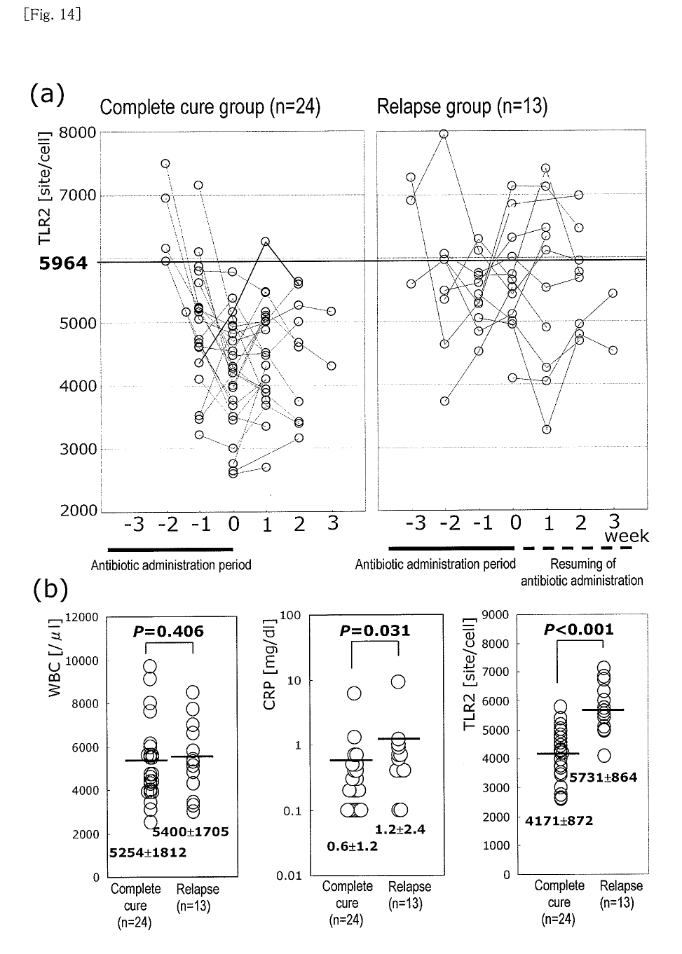

The TLR2 count of each patient with bacterial infection was determined by use of a peripheral blood sample collected from the patient during antibiotic administration or after completion of several-week antibiotic administration. The patients with bacterial infection (51 patients) include cases in which remission occurred at the time of initiation of treatment, antibiotic administration was terminated thereafter, and complete cure of bacterial infection was confirmed through follow-up after termination of antibiotic administration (until week 3 after termination of antibiotic administration); cases in which relapse occurred; cases in which remission did not occur, and amelioration and aggravation were repeated throughout the period of the disease; and cases in which the disease was aggravated into more serious bacterial infectious conditions. In each patient, among data on TLR2 count as obtained by the present quantitative determination method during the period of the disease (until complete cure or relapse was observed in the case in which remission occurred), the maximum TLR2 count was selected and compared with the TLR2 count of each healthy subject. The results are shown in FIG. 11. As shown in FIG. 11, in the patients with bacterial infection, during the period of the disease, TLR2 count may statistically significantly exceed the normal range. In some cases, TLR2 count was found to be relatively high, whereas in some cases, TLR2 count was found to fall within the normal range during the period of the disease. Clinical presentations indicated that the former cases included many patients (in particular, relatively young patients among patients aged 50 years or older and younger than 90 years) with prolonged bacterial infection (severe conditions continued for about one week or longer), and that the latter cases included many patients who were rapidly ameliorated and cured through administration of a first-choice antibiotic. The relationship between FIG. 10 and the corresponding clinical presentations may be otherwise described as the relationship between FIG. 13 and examination of efficacy of antibiotics in the treatment of infection in Example 12, or the relationship between FIG. 14 and examination of relapse (recurrence) in Example 13, to thereby clarify the clinical implication of the number of TLR2 sites per monocyte as determined during antibiotic administration.

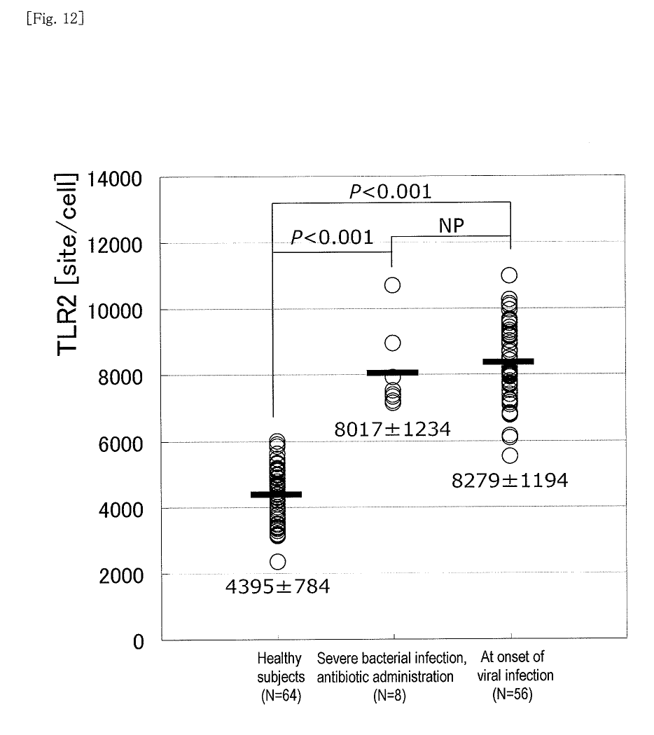

FIG. 12 shows comparison in TLR2 count between healthy subjects, patients (aged 50 years or older and younger than 90 years) with severe bacterial infection (including prolonged, severe infection cases, and sepsis/septic shock cases), and patients with viral infection, wherein the TLR2 count of each patient with bacterial infection corresponds to that as determined at the peak of the disease, and the TLR2 count of each patient with viral infection corresponds to that as determined at the onset of the disease.

There were selected eight patients (aged 50 years or older and younger than 90 years) with severe bacterial infections (including prolonged, severe, and hard-to-treat infection cases, and sepsis/septic shock cases); i.e., very severe patients in whom the effect of an antibiotic administered thereto was not envisaged. A multiple comparison test was carried out for comparing the TLR2 count of the hard-to-treat patient group with that of a group of healthy subjects or a group of patient with viral infections. As a result, the TLR2 count of the hard-to-treat patient group was found to be significantly different from that of the healthy subject group. Also, it was found to be difficult to obverse a significant difference between the TLR2 count of the hard-to-treat patient group and that of the group of patients with viral (mycotic) infection as determined at the onset of the disease.

However, in the case of the aforementioned hard-to-treat patients (serious infection patients), even if improved findings are observed through, for example, diagnostic imaging or a technique employing a blood marker after, for example, administration of a replaced antibiotic, when TLR2 count is maintained at high level after the peak of the disease, the patients are diagnosed with bacterial infection, and also, there is indicated a possibility that the patients suffer mixed bacterial-viral (or mycotic) infection, or a complication of bacterial infection and viral infection (or mycotic infection) has newly occurred during the period of bacterial infection. Thus, quantitative determination of TLR2 count has a great clinical significance, since it provides an opportunity for diagnostic review or information about mixed infection. Indeed, the present inventors experienced a case similar to that described above, in which a complication of mycotic Candida pneumonia was indicated on the basis of the fact that the level of TLR2 expression was maintained high, although amelioration of pneumonia was observed through chest X-ray radiography after the treatment with an antibiotic. In view of the foregoing, it will be understood how monitoring of the clinical course of a patient with infection by the present quantitative determination method provides useful information about the patient.

In patients (aged about 90 years or older) with bacterial infection (serious bacterial infection cases or serious septic cases), an increase in TLR2 count tended to be somewhat reduced, as compared with the case of patient with bacterial infections aged younger than about 90 years (in the case of such aged patients, the upper limit of TLR2 count was about 8,000 sites/cell as shown in FIG. 10).

### Example 12

### Examination of Efficacy of Drug in Bacterial Infection

FIG. 13 shows the results of time-course analysis of TLR2 count in 39 patient with bacterial infections receiving an antibiotic (therapeutic drug), the analysis being performed from the viewpoint of the effect of the antibiotic. Infection cases in which rapid amelioration or dramatic therapeutic response was observed one to three days after initiation of administration of the antibiotic are classified as a group of “marked effect.” Infection cases in which the antibiotic exhibited efficacy (although weak) are classified as a group of “weak effect,” from the viewpoint of efficacy of the antibiotic. As used herein, “weak effect” refers to a case in which, although data were unstable, reduction in level of an inflammatory marker (e.g., WBC or CRP) or slow improvement of systemic conditions was observed through follow-up over about one week. “No effect” refers to a case in which no tendency toward improvement was observed even one week after initiation of administration of the antibiotic. The serious bacterial infection cases (prolonged, severe infection cases) or hard-to-cure cases with sepsis/septic shock described above in Example 11-2 are classified as a group of “no effect,” from the viewpoint of response to the antibiotic treatment, since virtually no efficacy of the administered antibiotic was determined at the time of occurrence of such severe infection. The TLR2 count of each patient with bacterial infection shown in FIG. 13 was determined before examination of the efficacy of the antibiotic administered (i.e., determined by use of a blood sample collected within a period of time after initiation of administration of the antibiotic (day 2 to week 1)).

As shown in FIG. 13, when an antibiotic is administered to a patient, and then the TLR2 count of the patient is determined on day 2 or later, changes in clinical conditions (including WBC count and CRP level) of the patient can be predicted to some extent. In the case where a pathogen is a drug-resistant pathogenic bacterium (i.e., the most problematic clinical case), the number of drugs exhibiting efficacy (although weak) is limited, and a specific antibiotic must be employed. Therefore, in many cases, the efficacy of a drug cannot be determined until one week or more after initiation of administration of the drug to a patient. When the drug exhibits virtually no effect until determination of the efficacy of the drug, the drug merely grow bacteria during this period (one week), and the patient is in a serious state. Thus, it is very important to rapidly determine the efficacy of an antibiotic (drug) within a short period of time after initiation of administration of the antibiotic (i.e., from day 2 to week 1).

Also, as shown in FIG. 13, TLR2 count as determined within a short period of time after initiation of administration of an antibiotic can be employed as an index for determining the efficacy of the antibiotic.

Now will be specifically described one case in which application of the present quantitative determination method was useful. Before the patient was transferred, for the purpose of rehabilitation, to the medical institution in which the present inventors work, the patient had been treated for aspiration pneumonia in another institution and diagnosed as being completely cured after a sufficient period of follow-up. Immediately after admission to the medical institution in which the present inventors work, the patient developed bacterial pneumonia, and a cephem antibiotic was administered to the patient. Three days thereafter, the TLR2 count of the patient was found to be high (TLR2=7,499 sites/cell), and thus the antibiotic was determined not to be effective for the patient. Subsequently, the antibiotic was replaced by a carbapenem antibiotic, and, three days thereafter, the TLR2 count was found to be reduced to 5,197 sites/cell. Then, systemic conditions of the patient were rapidly improved. Finally, this case was diagnosed not with community-acquired pneumonia, but with relapse of aspiration pneumonia. Thus, monitoring of TLR2 count over time is very important for determining the efficacy of an antibiotic employed thereafter. Such monitoring realizes considerable reduction of an antibiotic ineffective for bacterial infection, rapid replacement of the ineffective antibiotic by an effective antibiotic before observation of excessive subjective symptoms (including fever) by a patient, and shortening of the period required for complete cure of the disease.

### Example 13

### Detection of Latent Infection Through Time-Course Assay of Samples from Patient with Bacterial Infections by Use of TLR2-Bound Standard Beads

### <Prediction of Relapse (Recurrence)>

Thirty-seven patients with bacterial infections (21 males and 16 females, age: 30 to 95) who consented to the use of their samples were subjected to treatment until they were in a remission phase. For the patients with bacterial infections, the present quantitative determination method was carried out throughout the period of the disease (including the period of antibiotic administration, and week 3 after termination of antibiotic administration), and follow-up was carried out for the purpose of determining whether or not relapse occurs after termination of antibiotic administration. All the 37 patients were diagnosed as being in a remission phase temporarily and allowed not to receive the antibiotic. Remission of the disease was determined on the basis of the physical findings and the blood test results (e.g., WBC count and CRP level), rather than TLR2 count. At the time of termination of antibiotic administration, WBC count fell within the normal range, and CRP level was generally maintained within the normal range. If possible, the level of TLR2 expression on a monocyte was determined once a week over the period from initiation of antibiotic administration to week 3 after termination of antibiotic administration. On the basis of the results of follow-up after termination of antibiotic administration, the patients in the remission phase of bacterial infection after antibiotic administration were classified into a group of “complete cure” (15 males and 9 females (total: 24 patients), age: 30 to 92 (mean: 65)) and a group of “relapse” (6 males and 7 females (total: 13 patients, 35.1%), age: 78 to 95 (mean: 88)).

Conceivably, in the relapsed patients, aggravation occurred as a result of growth of infecting bacteria which had remained (due to failure of growth inhibition) at the time of termination of antibiotic administration, or variation occurred in pathogenic bacteria in association with microbial substitution caused by antibiotic administration. The normal range of WBC count was determined to be 9,700/μL (upper limit) or less for male, or 9,300/μL (upper limit) or less for female. The normal range of CRP level was determined to be less than 0.5 mg/dL. At the time of termination of antibiotic administration, in all the 37 patients, WBC count fell within the normal range, but in some patients, CRP level exceeded the normal range. In order to determine whether or not CRP level is used as a factor for predicting relapse of infection, the 37 patients were classified into three groups on the basis of CRP level, and the relationship between risk of relapse and CRP level was examined. The results are shown in Table 7. As shown in Table 7, relapse was observed in six (26.1%) of 23 patients exhibiting a normal CRP level. Also, relapse was observed in five (50.0%) of 10 patients exhibiting a CRP level of 0.5 mg/dL or more and less than 1.0 mg/dL, and in two (50.0%) of four patients exhibiting a CRP level of 1.0 or more. In all the 37 patients, percent relapse was found to be 35.1% ( 13/37). Statistical analysis employing Fisher's exact test (extended) showed that percent relapse does not depend on CRP level (P=0.351). Thus, CRP level as determined at the time of termination of antibiotic administration, at which WBC count fell within the normal range, was found to be unsatisfactory as an index for predicting complete cure of bacterial infection.

TABLE 7

| | CRP level (mg/dL) | | |
|---|---|---|---|
| | <0.5 | 0.5-1.0 | >1.0 |
| Percent relapse (%) | 26.1% | 50.0% | 50.0% |
| (n/N) | (6/23) | (5/10) | (2/4) |

n = the number of relapsed patients, N = the number of tested patients

Meanwhile, on the basis of TLR2 count as determined at the time of termination of antibiotic administration, the 37 patients were classified into groups divided by the following TLR2 counts: 4,395 sites/cell (i.e., the average TLR2 count of healthy subjects); 5,179 sites/cell (i.e., the average TLR2 count+1×standard deviation); and 5,964 sites/cell (i.e., the average TLR2 count+2×standard deviation). In the patients exhibiting a TLR2 count lower than the average TLR2 count, percent relapse was found to be 6.7% ( 1/15), whereas in the patients exhibiting a TLR2 count equal to or greater than (the average TLR2 count+2×standard deviation), percent relapse was found to be very high (100%). In the patients exhibiting a TLR2 count equal to or greater than the average TLR2 count and lower than (the average TLR2 count+1×standard deviation), percent relapse was found to be 27.3% ( 3/10), whereas in the patients exhibiting a TLR2 count equal to or greater than (the average TLR2 count+1×standard deviation) and lower than (the average TLR2 count+2×standard deviation), percent relapse was found to be 66.7% ( 4/6) (Table 8). Fisher's exact test (extended) showed that percent relapse of bacterial infection statistically significantly increases with TLR2 count as determined at the time of termination of antibiotic administration, and the risk of relapse increases in accordance with an increase in TLR2 count (P<0.001). In conclusion, the quantitative value determined for TLR2 on a monocyte as determined at the time of termination of antibiotic administration can be employed as an important factor for predicting an outcome (“complete cure” or “relapse”) after the treatment of infection.

TABLE 8

| | Number of expressed TLR2 sites | | | |
|---|---|---|---|---|
| | on a monocyte (sites/cell) | | | |
| | <4395 | 4395-5179 | 5180-5964 | >5964 |
| Percent relapse (%) | 6.7% | 27.3% | 66.7% | 100% |
| (n/N) | (1/15) | (3/11) | (4/6) | (5/5) |

n = the number of relapsed patients, N = the number of tested patients

FIG. 14(a) shows change over time in quantitative value determined for TLR2 on a monocyte of patients with bacterial infections, which represents follow-up of the patients during and after treatment thereof. Different patients exhibited considerably different changes in quantitative value determined for TLR2 on a monocyte. The average of TLR2 counts of 62 healthy subjects (31 males and 31 females, age: 30 to 94 (mean: 60)) was determined (4,395 sites/cell), and the upper limit of the normal range of TLR2 count was determined to be 5,964 sites/cell (i.e., the average TLR2 count+2×standard deviation) (97.5% of healthy subjects exhibit a TLR2 count equal to or lower than the thus-determined upper limit). Patterns of change in TLR2 count of the patients were analyzed by use of the thus-determined normal range.

In the patients of a group of “complete cure,” TLR2 count of high level was rapidly reduced to fall within the normal range, or TLR2 count as measured first after initiation of antibiotic administration fell within the normal range, and such a low-level TLR2 count was maintained for three weeks after termination of antibiotic administration. In contrast, in the patients of a group of “relapse,” TLR2 count was maintained at high level. In many relapsed cases, TLR2 count increased at the time of termination of antibiotic administration, although WBC count or CRP level (i.e., conventional inflammatory marker) lowered. In the 13 relapsed patients, antibiotic administration was resumed immediately after determination of relapse of infection within three weeks after termination of antibiotic administration. Data on reduction in TLR2 count after resuming of antibiotic administration are also plotted in FIG. 14(a).

FIG. 14(b) shows data on the WBC count, CRP level, and number of TLR2 sites per monocyte of both the cured patients and the relapsed patients. At the time of termination of antibiotic administration, no significant difference was observed in WBC count between the “complete cure” group and the “relapse” group. A slightly significant difference was observed in CRP level between these two groups (P=0.031). However, conceivably, this significant difference is attributed to the fact that the “relapse” group included patients with underlying disease of metastatic liver cancer or connective tissue disease (i.e., a continuous increase in CRP level irrelevant to bacterial infection). In contrast to the cases of these conventional inflammatory parameters, TLR2 count in the “relapse” group is significantly higher than that in the “complete cure” group (P<0.001) at the time of termination of antibiotic administration. These data indicate that, at the time of termination of antibiotic administration (as determined on the basis of physical findings or normal data on level of blood markers (e.g., WBC and CRP), when TLR2 count is equal to or greater than the aforementioned upper limit, there is a high possibility of relapse. In some cases, when administration of an antibiotic is continued or the antibiotic is replaced by another one, TLR2 count is reduced to the aforementioned upper limit or lower. In such cases, there is a high possibility that percent relapse is reduced. Thus, careful follow-up of change over time in TLR2 count is envisaged to contribute to the prevention or rapid treatment of relapse. In addition, such follow-up is envisaged to prevent unwanted long-term antibiotic administration, and to minimize occurrence of drug-resistant bacteria, thereby reduce cases causing severe iatrogenic infection. This indicates that quantitative analysis of the number of TLR2 sites per monocyte through the present quantitative determination method provides more important data for the treatment/diagnosis of patients with infection than those obtained by existing test methods.

### Example 14

### TLR2 Count as Determined Upon Surgery

Table 9 shows the results of quantitative determination of the TLR2 count of samples collected before and after surgery, as well as clinical data.

TABLE 9

| | Case | | | | | |
|---|---|---|---|---|---|---|
| | 1 | 2 | | 3 | 4 | 5 |
| Surgical disease | Bladder tumor | Appendicitis, | | Infective | Angina pectoris | Paroxysmal |
| | | ovarian tumor | | endocarditis | | ventricular |
| | | | | | | tachycardia |
| Surgical procedure | Transurethral | Laparoscopic surgery | | Mitral valve | Coronary artery | ICD |
| | | | | replacement | bypass surgery | implantation |
| Before/after | After | Before | After | Before | After | After |
| surgery | 2 weeks | 1 day | 3 weeks | 1 month | 2 weeks | 2 months |
| WBC | 4000 | 5500 | 5600 | 6500 | 13000 | 3800 |
| CRP | 1.0 | 1.1 | 1.9 | 0.5 | 3.28 | 0.05 |
| TLR2 | 7193 | 6095 | 4909 | 3678 | 3834 | 5368 |
| Presence/absence | Presence | Presence | Absence | Absence | Absence | Absence |
| of infection | | | | | | |

Data obtained after surgery by use of conventional inflammatory markers cannot be employed for diagnosis in, for example, case 2 or case 4. In contrast, the TLR2 count of a sample from a patient, which serves as an index, corresponds to the pathological conditions of the patient (e.g., sensitive response to infection symptoms such as fever in case 1). This indicates that quantitative determination of TLR2 count is useful for monitoring of postoperative infection.

### Example 15-1

### Differentiation Between Infectious Disease and Non-Infectious Disease by the Level of TLR2 Expression (1)

The present quantitative determination method was carried out on samples from patients with non-viral liver dysfunction (Table 10), patients with organ ischemic necrosis (Table 11), patients with chronic heart failure (Table 12), patients with connective tissue diseases (Table 13), and patients with cancers (Table 14). Cases 4 and 6 shown in Table 10 are identical to cases 3 and 1 shown in Table 16, respectively. Case 2 shown in Table 11 is identical to case 2 shown in Table 17. Case 3 shown in Table 13 is identical to case 1 shown in Table 18.

TABLE 10

| | Case | | | | | | | |
|---|---|---|---|---|---|---|---|---|
| | 1 | 2 | 3 | 4 | 5 | 6 | | 7 |
| Non- | Alcoholic | Drug- | Congestive | Drug- | Drug- | Cardiogenic shock | | Drug- |
| infectious | | induced | liver | induced | induced | | | induced |
| diseae | | | | | | | | |
| Infection | Absence | Immediately | Absence | Absence | 1 week | Absence | Presence | Immediately |
| | | after cure | | | after cure | | | after cure |
| Presence or | Absence | Immediately | Absence | Absence | 1 week | Absence | Presence | Immediately |
| absence of | | after | | | after | | | after |
| antibiotic | | termination | | | termination | | | termination |
| administration | | | | | | | | |
| WBC | 6200 | 5200 | 3300 | 4800 | 5200 | 10500 | 5100 | 3400 |
| CRP | 0.1 | 1.0 | 0.1 | 0.2 | 0.1 | 4.0 | 0.1 | 0.5 |
| GOT | 87 | 55 | 34 | 89 | 55 | 653 | 36 | 89 |
| GPT | 58 | 66 | 16 | 107 | 66 | 1413 | 33 | 72 |
| TLR2 | 5225 | 5230 | 4077 | 4977 | 4871 | 3750 | 5533 | 6727 |
| Follow-up of | No | Complete | No | No | Complete | No | Aggravation | Relapse |
| infection | | cure | | | cure | | | |

TABLE 11

| Case | 1 | 2 |
|---|---|---|
| Disease | Acute myocardial infarction | Cardiogenic shock |
| Acute phase/chronic | 2 hours after onset | Day 2 after onset |
| phase | | |
| Presence or absence | Absence | Absence |
| of infection | | |
| Presence or absence | Absence | Absence |
| of antibiotic | | |
| WBC | 9600 | 10600 |
| CRP | 0.1 | 4.0 |
| TLR2 | 4241 | 3730 |

TABLE 12

| | Case | | | | | |
|---|---|---|---|---|---|---|
| | 1 | 2 | 3 | | 4 | 5 |
| Underlying | Paroxysmal | Myocarditis | Paroxysmal atrial | | Post- | Post- |
| disease | atrial fibrillation | Paroxysmal | fibrillation Tachycardia | | myocardial | myocardial |
| | Tachycardia | ventricular | | | infarction | infarction |
| | | tachycardia | | | | |
| Presence or | Absence | Absence | Absence | Acute | Acute | Acute |
| absence of | | | | pneumonia | bacterial | pneumonia |
| infection | | | | | enterocolitis | |
| Presence or | Absence | Absence | Absence | Absence | Presence | Presence |
| absence of | | | | | | |
| antibiotic | | | | | | |
| administration | | | | | | |
| WBC | 5600 | Not | 3300 | 5100 | 7400 | 5500 |
| | | determined | | | | |
| CRP | 0.05 | Not | 0.1 | 0.4 | 8.2 | 0.4 |
| | | determined | | | | |
| BNP | 1050 | Not | 1959 | Not | Not | 428 |
| | | determined | | determined | determined | |
| TLR2 | 1762 | 4693 | 4077 | 7270 | 5754 | 4930 |

TABLE 13

| | Case | | | | | | |
|---|---|---|---|---|---|---|---|
| | 1 | | 2 | | 3 | 4 | 5 |
| Underlying | Suspicion of | | Rheumatoid arthritis | | SLE | Rheumatoid | Sjogren's |
| disease | connective tissue | | | | | arthritis | syndrome |
| | disease | | | | | | |
| Presence or | Absence | Presence | Absence | Presence | Absence | Presence | Absence |
| absence of | | | | | | | |
| infection | | | | | | | |
| WBC | 5300 | 8300 | 5300 | 6000 | 4800 | 5800 | Not |
| | | | | | | | determined |
| CRP | 1.5 | 2.1 | 0.1 | 6.1 | 2.4 | 7.9 | Not |
| | | | | | | | determined |
| TLR2 | 4593 | 5959 | 3886 | 5211 | 3471 | 6567 | 4885 |

TABLE 14

| Case | 1 | | 2 |
|---|---|---|---|
| Underlying disease | Metastatic liver cancer | | Colon cancer |
| | (previous surgery of colon cancer) | | (before |
| | | | surgery) |
| Presence or absence | Negative | Presence | Absence |
| of infection | | | |
| WBC | 8900 | 6200 | 8400 |
| CRP | 14.9 | 11.3 | 0.5 |
| TLR2 | 5483 | 6471 | 3162 |

In the aforementioned patients, TLR2 count was found to be high in the presence of infection, or to fall within the normal range in the absence of infection. This indicates that TLR2 count can be employed for detection of infection in the patients. When a patient with a non-infectious underlying disease also develops infection, in many cases, the infection fails to be detected by use of conventional markers. In contrast, quantitative determination of TLR2 count realizes prompt action for the infection and proper treatment thereof, to thereby avoid risk to life.

### Example 15-2

### Differentiation Between Infectious Inflammatory Disease and Non-Infectious Inflammatory Disease (2)

When a conventional inflammatory marker (WBC or CRP) is employed in patients with a non-infectious inflammatory disease as described below, in many cases, the marker responds to the disease itself, and thus the disease is difficult to differentiate from an infectious inflammatory disease. However, the quantitative value determined for TLR2 on a monocyte barely responds to such a non-infectious inflammatory disease; i.e., the quantitative value determined for TLR2 on a monocyte does not increase in the presence of the disease. This feature of the number of TLR2 sites per monocyte provides very useful clinical information, which will next be described with reference to specific clinical cases.

(1) Detection of Infection Before and after Surgery

TABLE 15

| | Blood | | | | | | |
|---|---|---|---|---|---|---|---|
| Case | collection date | Diagnosis/condition | Age | Sex | TLR2 | WBC | CRP |
| 1 | 2006. 3. 1 | Intestinal ileus/enterocolitis | 85 | F | 5722 | 8900 | 2.1 |
| | | (recovery phase) | | | | | |
| | 2006. 3. 8 | Intestinal ileus/enterocolitis | | | 6095 | 5500 | 1.1 |
| | | (almost complete cure) | | | | | |
| | 2006. 3. 9* | (Emergency surgery) | | | | | |
| | | Appendicitis/pyometra | | | | | |
| | 2006. 3. 28 | After surgery | | | 4909 | 5600 | 1.9 |
| | | (remission phase) | | | | | |
| 2 | 2005. 11. 22 | Angina pectoris/surgical invation | 65 | M | 3834 | 13000 | 3.3 |
| | | (no infection) | | | | | |
| 3 | 2006. 9. 26 | Mitral insufficiency | 66 | F | | | |
| | | Dilated heart failure (surgery) | | | | | |
| | 2006. 12. 13 | CRTD implantation | | | | | |
| | 2007. 1. 15 | Onset of bacterial infection | | | 5927 | 10560 | 4.86 |
| | | (infected organ unknown) | | | | | |
| | 2007. 2. 27 | Infection | | | 5636 | 7030 | 0.87 |
| | | (remission phase) | | | | | |
| | 2007. 3. 6 | Bacteremia | | | 7922 | 10160 | 2.05 |
| | | (fever) | | | | | |
| | 2007. 3. 15 | Bacteremia | | | 5624 | 7890 | 0.55 |
| | | (remission phase) | | | | | |

Next will be described, with reference to detailed clinical courses of surgical patients (from hospitalization to complete cure of bacterial infection), actual cases (Table 15) in which quantitative determination of TLR2 count provided, in the field of surgical treatment, such useful patient information that cannot be obtained by conventional test methods.

Case 1: a patient who was hospitalized on Jan. 28, 2006 (physical findings: fever and marked abdominal bloating). A considerable increase in blood inflammatory marker level was observed (WBC: 26,300/μL, CRP: 27.4 mg/dL). Abdominal X-ray radiography showed marked accumulation of large bowel gas and small bowel gas. The patient was diagnosed with intestinal ileus and bacterial enterocolitis. Administration of a potent drug for improving intestinal peristaltic movement was initiated, and antibiotics were administered for bacterial enterocolitis. Several days thereafter, intestinal movement tended to be improved under fasting with intravenous hyperalimentation. On Mar. 7, 2006, the patient was in a clinical condition without fever and was diagnosed as being generally in a remission phase of intestinal ileus and bacterial enterocolitis (WBC: 5,500/μL, CRP: 1.1 mg/dL). During this period, two antibiotics were administered through injection in combination with oral administration of an antibiotic. On Mar. 8, 2006, the patient was determined to be almost completely cured of bacterial infection, and the patient was transferred to a gastrointestinal hospital for the purpose of close examination for the cause of intestinal ileus (suspicion of intestinal tumor, etc.). In the hospital, the patient was found to have appendicitis and subjected to emergency surgery. Since the patient was also found to have pyometra during laparotomy, the diseased portion was also excised. On Mar. 24, 2006, the patient was transferred to the medical institution in which the present inventors work after surgery and a period of stable clinical conditions. WBC count was found to be 7,700/μL, and CRP level was found to be as high as 7.7 mg/dL. This was considered to be attributed to an increase in blood inflammatory marker level due to surgical invasion, as well as a possibility of residual bacterial infection. On Mar. 28, 2006, the level of blood inflammatory markers was found to be still high (WBC: 5,600/μL, CRP: 1.9 mg/dL), and a possibility of residual bacterial infection was not completely excluded. On Apr. 4, 2006, systemic conditions of the patient were improved (WBC: 4,100/μL, CRP: 0.4 mg/dL), and the patient was determined to be completely cured of infection.

On Mar. 8, 2006, on which the patient was determined to be almost completely cured of bacterial infection on the basis of a tendency toward reduction in level of conventional blood inflammatory markers, as well as the clinical conditions of the patient, TLR2 count was found to exceed the normal range and to fall within a range corresponding to infection (6,095 sites/cell), which was higher than that determined one week before. These data indicated that the patient was not completely cured of infection and predicted aggravation of the disease. Indeed, as a result of close examination in the gastrointestinal hospital, the patient was found to have surgically applicable appendicitis, and during laparotomy, the patient was found to have pyometra (infection). Subsequently, on Mar. 28, 2006, the level of conventional blood inflammatory markers was found to be still high (WBC: 5,600/μL, CRP: 1.9 mg/dL) about 20 days after surgery, and difficulty was encountered in differentiating between an increase in blood inflammatory marker level due to surgical invasion, and a possibility of residual bacterial infection. However, at this point in time, TLR2 count was determined to be 4,909 sites/cell (normal range). Thus, quantitative determination of TLR2 count, which is not affected by surgical invasion, provided information about complete cure of bacterial infection. Thereafter, conditions of the patient or data on conventional blood inflammatory marker level showed a tendency toward complete cure. This indicates utility of quantitative determination of TLR2 count.

Case 2: a patient who underwent coronary artery bypass thoracotomy for angina pectoris (at week 2). Although the levels of conventional inflammatory markers (WBC and CRP) were found to be clearly high, TLR2 count was determined to be 3,834 sites/cell (normal range). At this point in time, in consideration of no antibiotic administration (as described above, even in the case of bacterial infection, TLR2 count is affected by the efficacy of an antibiotic employed therefor), the patient was determined to have no infection. Indeed, the subsequent clinical course of the patient did not show infection-associated conditions.

Case 3: a patient who underwent surgery twice and in whom the level of inflammatory markers (WBC and CRP) increased after surgery, which increase was caused by infection, rather than by surgically invasive factors (including allergy to an implanted medical device). The patient was in a severe heart failure state with dilated cardiomyopathy and mitral insufficiency, and surgery was carried out on Sep. 26, 2006. Valvuloplasty was carried out for mitral insufficiency, and left ventricular reduction surgery was carried out for dilated cardiomyopathy. A protein-coated patch was employed for left ventricular reduction surgery. Subsequently, on Dec. 13, 2006, implantation of a cardiac resynchronization therapy defibrillator (CRTD) was carried out for the purpose of improvement of severe heart failure. On Jan. 15, 2007, WBC count and CRP level were found to increase. Since the patient was considered to be easily infected with bacteria, an antibiotic was administered to the patient, but diagnosis was not established. On Jan. 15, 2007, an increase in WBC count was observed, but typical peripheral blood data for diagnosis of bacterial infection were not obtained (no increase in neutrophil count and an increase in eosinophil were observed). Therefore, infection, surgical invasion, allergy to the implanted medical device, etc. were selected as factors for differentiation. On Jan. 15, 2007, TLR2 count was determined to be 5,927 sites/cell; i.e., TLR2 count was found to almost fall within a range corresponding to infection. Thereafter, an antibiotic was administered to the patient through injection for one week, and then the patient exhibited stable symptoms. Since the effect of the antibiotic was observed, the patient was suspected of having infection. Subsequently, on Feb. 15, 2007, WBC count and CRP level were found to generally fall within the respective normal ranges, but TLR2 count was determined to be 5,636 sites/cell. Therefore, there was suggested a high possibility of relapse (66.7%) in consideration of the results of the aforementioned examination of percent relapse of bacterial infection (Example 13) (data shown in Table 8). On Feb. 27, 2007, WBC count and CRP level were found to increase again, and TLR2 count was determined to be high (7,922 sites/cell), which fell within a range corresponding to infection. At this point in time, a blood culture test was carried out. At a later date, Gram-positive bacteria were detected, and bacterial infection (bacteremia) was also detected. Thus, relapse was confirmed clinically.

The above description of the three cases specifically indicates how it is difficult to determine the presence or absence of postoperative infection through conventional, general test methods. Also, the description shows that TLR2 count is a useful index for such postoperative follow-up.

(2) Hepatic Disorder

TABLE 16

| | Blood | | | | | | | | |
|---|---|---|---|---|---|---|---|---|---|
| | collection | | | | | | | | |
| Case | date | Diagnosis | Age | Sex | TLR2 | WBC | CRP | AST | ALT |
| 1 | 2005. 12. 13 | Hypoxic hepatic | 84 | F | 3750 | 10500 | 4.0 | 653 | 1413 |
| | | disorder | | | | | | | |
| 2 | 2006. 4. 19 | Alcoholic hepatic | 73 | M | 5225 | 6900 | 0.1 | 87 | 58 |
| | | disorder | | | | | | | |
| 3 | 2006. 2. 13 | Chronic drug- | 79 | F | 4977 | 4800 | 0.2 | 89 | 107 |
| | | induced hepatitis | | | | | | | |
| 4 | 2006. 11. 30 | Hepatitis B | 42 | M | 4135 | 4000 | 0.1 | 24 | 26 |
| | | carrier | | | | | | | |
| 5 | 2006. 2. 14 | Chronic hepatitis | 75 | F | 3800 | 3800 | 0.1 | 40 | 55 |
| | | B and C | | | | | | | |

Table 16 shows patients with hepatic disorder and no infection, patients carrying hepatitis virus having no growth activity, and patients with chronic hepatitis in which viral growth activity is very low. As shown in Table 16, in the cases of hepatic disorder with no infection, hepatitis virus carrier, and chronic hepatitis, no increase in TLR2 count was observed. These data indicate that when such a patient develops acute infection, TLR2 count can be employed as a useful index for monitoring the conditions of the patient. As described above, cases 1 and 3 shown in Table 16 are identical to cases 6 and 4 shown in Table 10, respectively.

(3) Organ Ischemic Necrosis Such as Myocardial Infarction or Cerebral Infarction

TABLE 17

| | Blood | | | | | | | | |
|---|---|---|---|---|---|---|---|---|---|
| | collection | | | | | | | | |
| Case | date | Diagnosis | Age | Sex | TLR2 | WBC | CRP | AST | ALT |
| 1 | 2005. 12. 13 | Acute cerebral | 92 | F | 5469 | 6500 | 1.5 | 30 | 24 |
| | | infarction | | | | | | | |
| 2 | 2006. 4. 19 | Acute myocardial | 50 | M | 4241 | 9600 | 0.1 | 50 | 20 |
| | | infarction | | | | | | | |
| 3 | 2007. 2. 13 | Acute cerebral | 77 | F | 3540 | 7100 | 0.1 | 46 | 30 |
| | | infarction | | | | | | | |
| 4 | 2005. 12. 13 | Ischemic hepatic | 84 | F | 3750 | 10500 | 4.0 | 653 | 1413 |
| | | disorder | | | | | | | |
| | 2005. 12. 26 | Bacterial pneumonia | | | 7228 | 5100 | 0.1 | 36 | 33 |
| | | complication | | | | | | | |

Cases 1 to 3 shown in Table 17 are patients with acute ischemic organ necrosis. The level of TLR2 expression on a monocyte did not respond to organ ischemic necrosis and fell within the normal range. In case 4, the disease was developed on Dec. 10, 2005. TLR2 count was followed up over several days after the onset of the disease, and as a result, the thus-determined TLR2 count was found to fall within the normal range. This indicate that the quantitative value determined for TLR2 on a monocyte does not respond to organ ischemic necrosis, so long as intercurrent infection does not occur (TLR2 count was found to be as high as 7,228 sites/cell on Dec. 26, 2005, and then bacterial pneumonia was developed on Jan. 2, 2006). These data indicate that the profile of TLR2 count differs from that of the level of a conventional inflammatory marker (WBC or CRP), which increases in response to ischemic organ necrosis itself. Thus, quantitative determination of TLR2 count can differentiate intercurrent infection at an early stage. As described above, case 2 shown in Table 17 is identical to case 2 shown in Table 11.

(4) Connective Tissue Disease

TABLE 18

| | Blood | | | | | | |
|---|---|---|---|---|---|---|---|
| Case | collection date | Diagnosis | Age | Sex | TLR2 | WBC | CRP |
| 1 | 2005. 6. 7 | Systemic lupus erythematosus | 78 | F | 3471 | 4800 | 2.4 |
| 2 | 2006. 3. 15 | Rheumatoid arthritis | 83 | M | 4540 | 6000 | 6.1 |
| 3 | 2006. 12. 26 | Rheumatoid arthritis | 74 | F | 4374 | 5300 | 0.1 |
| 4 | 2007. 12. 18 | Aortitis syndrome | 72 | F | 3074 | 12500 | 11.1 |
| 5 | 2007. 2. 26 | RS3PE | 90 | F | 4918 | 8400 | 8.9 |
| 6 | 2007. 3. 26 | Raynaud's syndrome | 56 | M | 3900 | 4500 | 0.1 |
| 7 | 2007. 2. 7 | Pustular psoriasis | 57 | M | 4958 | 7350 | 6.22 |

RS3PE: Remitting symmetric seronegative synovitis with pitting edema

Table 18 shows patients with various types of connective tissue diseases. Connective tissue disease itself causes an increase in level of CRP (i.e., a conventional inflammatory marker), as well as infection-like subjective symptoms (e.g., fever and arthralgia). In some cases of connective tissue disease (e.g., case 4), WBC count also increases. The difference between CRP level and WBC count (i.e., on the basis of the fact that an increase in CRP level is not generally correlated with an increase in WBC count) may be used as an index for differentiation between connective tissue disease and infection. However, such a differentiation is not necessarily applied to all cases of connective tissue disease. In many cases, patients with connective tissue disease always receive, for example, a low dose of steroid or an immunosuppressive drug, and the patients are readily infected with bacteria. When a patient with connective tissue disease also develops infection, a continuous increase in level of a conventional inflammatory marker makes rapid diagnosis of infection difficult. In addition, whether or not such a patient is cured of infection in a remission phase is determined on the basis of doctor's experience. Thus, TLR2 count can be employed as a useful objective index for determining complete cure of infection. As described above, case 1 shown in Table 18 is identical to case 3 shown in Table 13.

(5) Malignant Tumor

TABLE 19

| | Blood | | | | | | |
|---|---|---|---|---|---|---|---|
| | collection | | | | | | |
| Case | date | Diagnosis | Age | Sex | TLR2 | WBC | CRP |
| 1 | 2006. 4. 5 | Metastatic | 85 | F | 5458 | 8900 | 14.9 |
| | | liver cancer | | | | | |
| | | (end-stage) | | | | | |
| | 2006. 5. 24 | Cholecystitis | | | 7313 | 14900 | 13.6 |
| | | complication | | | | | |
| 2 | 2005. 6. 7 | Colon cancer | 86 | M | 3162 | 8400 | 2.4 |
| | | (before | | | | | |
| | | surgery) | | | | | |

Table 19 shows cancer patients. Case 1: a terminal-stage cancer patient who was determined not to receive active cancer therapy by a specialist. In the patient, intercurrent infection was very difficult to diagnose, since tumor fever (about 38° C.) was continued, and CRP level was maintained high. Even when, for example, an antibiotic was experimentally administered to the patient on the basis of fever, defervescence was not observed, and therefore the cancer was determined to be less associated with bacterial infection. Thus, the presence or absence of infection had to be determined through experimental antibiotic administration, and follow-up of objective findings and subjective symptoms. Since there was no objective index for determining aggravation or remission of infection, quantitative determination of TLR2 expression level was initiated in case 1. As a result, TLR2 count was found to fall within the normal range at the period when no infection was observed and antibiotic administration was determined to be able to be terminated. Tumor fever (about 38° C.) was intermittently observed, but did not continue for several days. Thereafter, intermittent fever changed into continuous fever at the period when TLR2 count increased, which period corresponded to the period when the presence of infection was determined. No continuous fever was observed after antibiotic administration. This case indicates that, even in a patient with tumor fever, quantitative determination of TLR2 count provides useful information about intercurrent infection. Since the infection-induced fever (other than tumor fever) of a patient is reduced to a minimum possible extent through treatment on the basis of TLR2 count, suffering of the patient can be alleviated.

Case 2: a patient who was hospitalized with a complication of colon cancer with bacterial enterocolitis. Due to severe infectious conditions of the patient, cancer extension failed to be examined. Therefore, firstly, infection was treated, followed by close examination, and cancer treatment was carried out. In this patient, close examination was carried out on Jun. 7, 2005, on which infection was considered to be sufficiently inhibited on the basis of TLR2 count and objective findings, and cancer tissue and lymph nodes were excised through surgery (laparotomy) to a maximum possible extent (although incomplete). The patient showed an uneventful postoperative course without infection and was discharged from the hospital.

As shown in the aforementioned two cases, no increase in TLR2 count was observed in common cancers (other than special cancers). This suggests that quantitative determination of TLR2 expression level has a great medical significance for diagnosis of such a cancer and for treatment thereof (including surgery). The expression “special cancers” is used herein for emphasizing that no increase in TLR2 count is not necessarily applied to all cancers, since, in some cancers (among various types of cancers), infection-like inflammation is highly likely to occur, and thus there is a high possibility of an increase in TLR2 count.

(6) Hematologic Disease

TABLE 20

| | Blood | | | | | | | | | |
|---|---|---|---|---|---|---|---|---|---|---|
| | collection | | | | | | | | | |
| Case | date | Diagnosis | Condition | Treatment | Age | Sex | TLR2 | WBC | CRP | G-CSF |
| 1 | 2006. 1. 10 | Sepsis, severe | Tendency | Change of | 95 | M | 7020 | 1900 | 1.0 | No |
| | | pneumonia/ | toward | antibiotic | | | | | | |
| | | myelodysplastic | aggravation | | | | | | | |
| | 2006. 1. 16 | syndrome | Recovery | Change of | | | 5500 | 5000 | 0.5 | Administration |
| | | | phase | antibiotic | | | | | | |
| | 2006. 1. 22 | | Remission | Termination of | | | 6393 | 5400 | 0.4 | Administration |
| | | | phase | administration | | | | | | |
| | 2006. 2. 1 | | Relapse of | Resuming of | | | 6265 | 5400 | 0.7 | Administration |
| | | | infection | antibiotic | | | | | | |
| | | | | administration | | | | | | |
| 2 | 2007. 11. 27 | Adult T-cell | Onset | No | 83 | F | 4009 | 6500 | 0.1 | No |
| | | leukemia | | | | | | | | |
| 3 | 2006. 12. 14 | Adult T-cell | Onset | Radiotherapy | 56 | M | 4843 | 2000 | 0.1 | No |
| | 2006. 12. 20 | leukemia | Infection | Ganciclovir | | | 7689 | 2400 | 1.2 | No |
| | | | complication | | | | | | | |
| | 2006. 12. 26 | | Amelioration | Ganciclovir | | | 3805 | 2100 | 0.6 | No |
| | | | of infection | | | | | | | |

Case 1 shown in Table 20: a patient who had an underlying disease of myelodysplastic syndrome, refractory sepsis, and refractory pneumonia, and in whom complete cure of the diseases was difficult despite long-term administration of various antibiotics. On Jan. 7, 2006, the patient developed bacterial pneumonia, and administration of an antibiotic (MINO) was initiated. On Jan. 10, 2006, the patient showed anemia, thrombocytopenia, and pancytopenia (WBC: 1,900/μL, CRP: 1.0 mg/dL). These symptoms were attributed to an underlying disease of myelodysplastic syndrome. In this patient, chest X-ray radiography showed a clear image of pneumonia, but severe bacterial infection failed to be determined by the WBC count (1,900/μL) as measured on Jan. 10, 2006. Systemic conditions of the patient showed no effect of the antibiotic MINO on bacterial pneumonia, and TLR2 count was determined to be high (7,020 sites/cell) at this point in time.

On the following day (i.e., January 11), the antibiotic MINO was replaced by combination of two antibiotics (CLDM and FOM). On January 18, high fever was not observed, but continuous slight fever was observed. Therefore, these antibiotics were replaced by the following two antibiotics (CAZ and ISP), and treatment for bacterial pneumonia was carried out. As a result, conditions of the patient were ameliorated, and an image of pneumonia tented to become unclear.

As shown in the aforementioned case of myelodysplastic syndrome (hematologic disease), in such a patient, WBC count is affected by the disease itself, and WBC count can no longer be used as an index for determining the severity of bacterial infection. However, even in the aforementioned case, in which the underlying disease caused leukopenia, the TLR2 count as measured on January 10 (7,020 sites/cell) provided information about the severity of infection, as well as no efficacy of the antibiotic MINO administered at this point in time. Thus, it was shown that, as examined above, TLR2 count is employed as an index for determining the efficacy of an antibiotic in such a patient with hematologic disease.

On January 11, injection of a therapeutic dose of G-CSF (i.e., a leukocytosis-promoting factor) was initiated for coping with leukopenia caused by myelodysplastic syndrome. Therefore, WBC count, which was affected by both myelodysplastic syndrome and G-CSF, failed to be used as an index for determining the severity of bacterial infection. From Jan. 20, 2006 (or thereabouts), the patient maintained a normal body temperature of 36.0 to 36.9° C. and was diagnosed as being in a remission phase. On Jan. 22, 2006, on the basis of the data (WBC: 5,400/μL, CRP: 0.4 mg/L), administration of the antibiotics was terminated, and follow-up of the patient was initiated. However, at this point in time, TLR2 count was determined to be as high as 6,393 sites/cell, which indicated that percent relapse would be high. On February 1, the patient showed a slight fever of 37.0 to 37.9° C. and tachypnea; i.e., relapse of bacterial infection was observed (WBC: 5,400/μL, CRP: 0.7 mg/dL).

Thus, it was shown that TLR2 count serves as an “index for determining relapse” as described above, even when WBC count is no longer used as an index for determining the severity of infection, because of pancytopenia caused by myelodysplastic syndrome (underlying disease), as well as administration of a therapeutic dose of G-CSF.

In case 2, TLR2 count was determined at the time of onset of adult T-cell leukemia (ATL). WBC count was found to be 6,500/μL (in peripheral hemogram, eosinophil: 0.0%, basophil: 2.0%, rod-shaped neutrophil: 1.5%, segmented neutrophil: 39.0%, lymphocyte: 13.5%, monocyte: 9.0%, and abnormal lymphocyte: 34.5%), and CRP level was found to be 0.1. This case indicated that TLR2 count responds to neither the state of carrying HTLV-I virus nor the onset of ATL. Conceivably, the reason why TLR2 count does not respond to the onset of ATL is attributed to the fact that, unlike the case of common viral infection, ATL—which is a virus-induced hematologic cancer (helper T-cells (Th1) of a host are infected with the virus, and the virus is integrated into the host DNA as a provirus)—is characterized by abnormal growth of ATL cells, rather than cell tissue destruction by abnormal growth of the virus. Case 3: a patient who developed ATL and also developed cytomegalovirus infection during radiotherapy for ATL. On Dec. 14, 2006, the patient was under radiotherapy and was diagnosed as having no intercurrent infection. On Dec. 20, 2006, the patient developed fever and upper respiratory tract infection (TLR2 count was determined to be as high as 7,689 sites/cell), and then the patient was diagnosed with cytomegalovirus pneumonia. Ganciclovir was administered to the patient for the treatment of cytomegalovirus infection, and the viral infection was rapidly ameliorated. On Dec. 26, 2006, TLR2 count was reduced to 3,805 sites/cell. These data indicate that TLR2 count can be employed for monitoring the treatment of viral infection. In cases 2 and 3, it was shown that TLR2 count does not change in response to ATL (adult T-cell leukemia) itself, which is a hematologic cancer, but TLR2 count increases in the presence of intercurrent infection.

The above-described hematologic disease cases specifically show that the level of a conventional inflammatory marker (WBC or CRP) is greatly affected by a hematologic disease itself, and thus the marker no longer serves as an index for determining the presence of infection. Even in such a case, the number of TLR2 sites per monocyte serves as described above. Specifically, as shown in the aforementioned cases of “malignant tumor,” TLR2 count can be used for determining the presence of infection at an early stage, and also can be used for monitoring response to treatment of the infection.

(7) Allergic Disease

TABLE 21

| | Blood | | | | | | |
|---|---|---|---|---|---|---|---|
| | collection | | | | | | |
| Case | date | Diagnosis | Age | Sex | TLR2 | WBC | CRP |
| 1 | 2006. 5. 24 | Asthmatic attack | 98 | F | 2450 | 7200 | 3.2 |
| 2 | 2005. 12. 12 | Asthmatic attack/bacterial pneumonia | 74 | F | 6290 | 6000 | 6.1 |
| | 2005. 12. 26 | Asthmatic attack | | | 4750 | 5300 | 0.1 |
| 3 | 2007. 2. 21 | Anaphylactic shock | 77 | F | 4737 | 22590 | 1.63 |
| 4 | 2007. 4. 11 | Drug-induced allergic rash | 83 | F | 5123 | 5100 | 2.37 |

Table 21 shows a case of asthmatic attack, a case of status asthmaticus caused by bacterial infection, a case of anaphylactic shock with severe allergic symptoms caused by a drug (Futhan), and a case of drug-induced allergic rash.

In case 1, TLR2 count was determined at the time when asthmatic attacks occurred, and the patient was diagnosed as not having intercurrent infection (including bacterial infection) by clinical follow-up. TLR2 count was found to fall within the normal range, and no increase in TLR2 count was observed.

Case 2: a patient who was hospitalized with a complication of asthmatic attack with bacterial infection. During the intercurrent bacterial infection, TLR2 count was found to increase to 6,290 sites/cell. The infection was completely cured after two-week continuous antibiotic administration, but repeated asthmatic attacks were observed at this point in time. Studies on the aforementioned two cases indicate that the quantitative value determined for TLR2 on a monocyte does not increase in patients with asthmatic attack.

Case 3: a patient who developed anaphylactic shock and eventually suffered a cardiac arrest. Blood collected from the patient was tested, and then steroid was administered to the patient. WBC count considerably increased, but no increase in TLR2 count was observed before steroid administration (high-dose steroid administration (e.g., pulse administration) inhibits the level of TLR2 expression: Pons J., et al. Respir. Res. 2006; 7: 64).

Case 4: a patient who developed allergic rash induced by oral administration of an antibiotic. A slight increase in CRP level was observed, and TLR2 count was determined at this point in time. The results are shown in Table 21. As is clear from these data, no increase in TLR2 count is observed in such a drug-induced rash case.

Since no increase in TLR2 count is observed in allergic disease, an intercurrent or complicating infection can be more specifically differentiated on the basis of an increase in TLR2 count. Thus, as shown above, TLR2 count can be employed as a useful index for determining infection in the field of allergic disease.

(8) Thyroid Disease

TABLE 22

| | Blood | | | | | | |
|---|---|---|---|---|---|---|---|
| | collection | | | | | | |
| Case | date | Diagnosis | Age | Sex | TLR2 | WBC | CRP |
| 1 | 2006. 12. 25 | Subacute | 24 | F | 7578 | 3100 | 2.7 |
| | | thyroiditis | | | | | |
| 2 | 2006. 11. 29 | Basedow's | 60 | F | 5270 | | |
| | | disease | | | | | |

In hyperthyroidism, thyroid hormone is excessively secreted, and blood thyroid hormone level is maintained high, resulting in hypermetabolism and development of various symptoms (e.g., palpitation, body weight reduction, and finger tremor). Hyperthyroidism-related diseases include Basedow's disease, Plummer's disease, and subacute thyroiditis. Basedow's disease is a typical hyperthyroidism-related disease, and Basedow's disease cases account for most hyperthyroidism cases. Basedow's disease develops in the presence of an antibody to thyroid-stimulating hormone (TSH) receptor of thyrocytes (which antibody serves as a thyroid-stimulating substance), wherein the thyroid gland is diffusely enlarged. As has been elucidated, Basedow's disease is related to genetic predisposition. Plummer's disease is caused by hyperfunctioning adenoma, wherein solitary adenoma is found. Subacute thyroiditis is also associated with hyperthyroidism. Subacute thyroiditis is caused by viral infection, wherein thyroid tissue destruction results in fever, thyroid pain, and transient thyrotoxicosis. These three types of diseases are considered very difficult to differentiate from one another.

As shown in Table 22 above, in the case of subacute thyroiditis (i.e., a hyperthyroidism-related disease), which is caused by viral infection, TLR2 count was found to be as high as 7,578 sites/cell. Therefore, subacute thyroiditis can be differentiated by virtue of this feature (i.e., high TLR2 count). In the case of Basedow's disease, which is classified as an autoimmune disease, similar to the aforementioned case of connective tissue disease, TLR2 count was found not to increase.

### Example 16

### Monitoring of Viral Infection

### <Influenza Virus Infection>

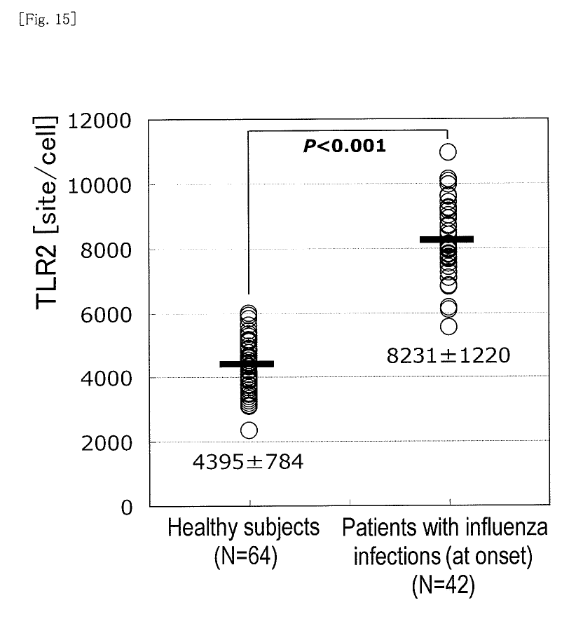

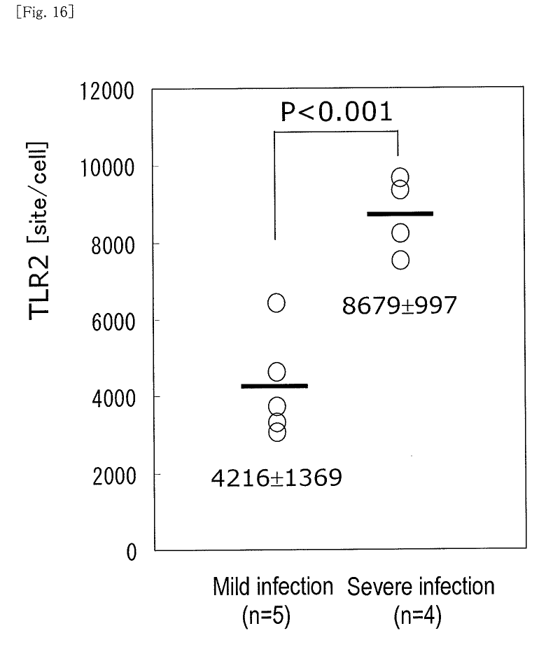

Similar to the cases of patient with viral infections shown in FIG. 10, the quantitative value determined for TLR2 on a monocyte of a group of 42 patients infected with influenza A and B viruses (18 males and 24 females, age: 9 to 93 (mean: 42)) was determined at the time of onset of influenza (i.e., at the time of consultation in hospital immediately after development of subjective symptoms of infection), and the thus-determined TLR2 count was compared with that of a group of healthy subjects. As shown in FIG. 15, the TLR2 count of the patient group was found to be such a high level that a cut-off value giving virtually no false-negative results can be provided between the TLR2 count of the patient group and that of the healthy subject group. Diagnosis of influenza infection was carried out by means of a rapid diagnosis kit using immunochromatography. FIG. 10 shows plots of data on TLR2 count as measured at the onset of viral infections (other than influenza virus infection). As shown in FIG. 16, similar to the case of influenza virus infection, TLR2 count was found to be very high in the case of common cold.

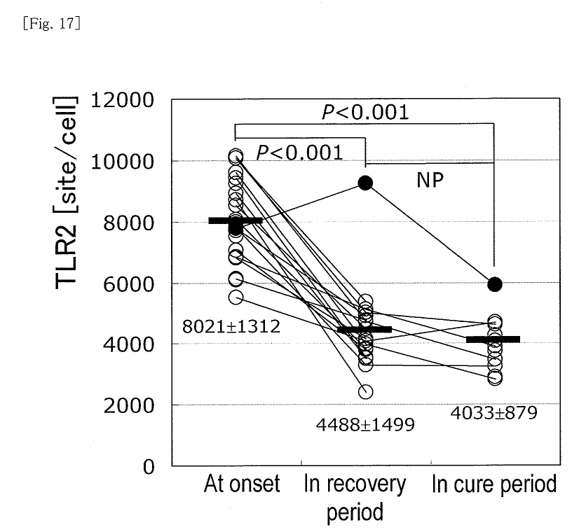

Subsequently, follow-up of the influenza A and B patients was carried out for about one month. The TLR2 count of each patient was determined at the time of the onset of influenza infection, in a recovery period (i.e., day 5 to day 14 after the onset of influenza infection), and in a cure period (i.e., day 15 or later after the onset of influenza infection). Quantitative determination of TLR2 count was carried out to a maximum possible extent. The results are shown in FIG. 17. In most patients, Tamiflu was administered for three to five days after the onset of the disease. In the recovery period, some patients exhibited influenza symptoms (e.g., cough and slight malaise), but most patients showed no subjective symptoms (i.e., nearly complete cure). In the subsequent cure period, no patients showed symptoms of influenza infection. Upon quantitative determination of TLR2 count for each patient, the WBC count, leukocyte fraction, CRP level, and biochemical data of the patient were examined for determining whether or not the patient developed a complication, as well as the presence or absence of subjective symptoms. In most cases (except for a case in which TLR2 count as measured one week after the onset of influenza infection was higher than that as measured at the time of the onset thereof), TLR2 count was reduced to fall within the normal range in the recovery period. In this exceptional case, in which TLR2 count was not reduced in the recovery period (i.e., the TLR2 count was higher than that as measured at the time of onset), WBC count and CRP level fell within the respective normal ranges, but proximal-muscle-dominant muscular weakness was observed in objective physical findings. The patient was suspected of having a disease induced by influenza virus infection, and thus close examination of the patient was carried out. As a result, the patient was diagnosed with inclusion body myositis. Thereafter, the conditions of the patient were ameliorated through steroid administration, and reduction in TLR2 count was observed in the cure period. Thus, it was shown that TLR2 count can be employed for monitoring the severity of a viral disease and the course of cure of the disease, and that a high TLR2 count can indicate the presence of a more advanced, severe disease. Therefore, quantitative determination of TLR2 expression level has a great clinical significance in the field of viral infection. Conceivably, monitoring of the severity of viral infection by use of quantitative TLR2 value is fully applicable to the case of new-type influenza (on the basis of the assumption that new-type influenza is classified into class V as in the case of influenza A or B, and defense response to new-type influenza is similar to that to influenza A or B).

FIG. 16 is a graph showing quantitative TLR2 values of common cold cases which were classified on the basis of the severity of the cases. In many viral infection cases (including influenza infection), acute and severe symptoms are shown at the time of onset thereof, and the severity thereof is difficult to evaluate clinically as in the case of bacterial infection. Unlike such viral infection cases, common cold is one of few viral infection cases showing symptoms which can be classified on the basis of severity. The reason why only data on common cold cases are presented in FIG. 16 is to show whether or not the degree of increase in quantitative TLR2 value-varies with the severity of viral infection. Specifically, as shown in FIG. 16, it can be determined that, in the case of mild viral infection, the degree of increase in quantitative TLR2 value is small, whereas in the case of severe viral infection, the degree of increase in quantitative TLR2 value is large. Table 23 shows profiles of nine subjects whose data are presented in FIG. 16.

TABLE 23
(Nine common cold cases)

| | | | | | | | | | | | Fluid |
|---|---|---|---|---|---|---|---|---|---|---|---|
| | | | | | Severe/ | Body | Systemic | Runny | | | replacement |
| Age | Sex | TLR2 | CRP | WBC | Mild | temp. | malaise | nose | Cough | Inappetence | therapy |
| 46 | F | 7512 | 0.1 | 5450 | Severe | 37.8 | + | + | + | + | + |
| 28 | M | 9669 | 6.5 | 8000 | Severe | 38.0 | + | + | + | + | + |
| 43 | M | 9328 | 1.3 | 8040 | Severe | 38.5 | + | + | + | + | + |
| 30 | F | 8210 | 2.3 | 5300 | Severe | 39.4 | + | + | + | + | + |
| 42 | M | 3291 | 0 | 5000 | Mild | 37.1 | − | + | − | − | − |
| 41 | F | 6413 | 0.35 | 7100 | Mild | 37.0 | − | + | + | − | − |
| 32 | F | 3719 | 0.05 | 7370 | Mild | 37.1 | − | + | + | − | − |
| 24 | F | 4628 | 0.2 | 7380 | Mild | 37.0 | − | + | − | − | − |
| 22 | F | 3033 | 0.12 | 7320 | Mild | 37.7 | + | − | + | − | − |

These common cold cases show that the degree of increase in quantitative TLR2 value varies with the clinical severity of viral infection. In addition, the aforementioned data indicate that quantitative TLR2 value is reduced with recovery from influenza infection, and thus quantitative TLR2 value can be employed as an index for monitoring the severity of viral infection or the degree of recovery after treatment. The clinical severity of viral infection is considered to be correlated with the growth activity of the virus.

### Example 17

### Cardiomyopathy (Including Cardiac Sarcoidosis)

The cause of sarcoidosis has not yet been fully elucidated, but sarcoidosis is proposed to be caused by infection; for example, there is a theory that sarcoidosis develops as a result of hypersensitive immunoreaction induced by endogenous infection with Propionibacterium acnes. Specifically, onset of sarcoidosis (i.e., a systemic granulomatous disease) may be triggered through endogenous activation (caused by environmental factors such as stress) of cell-wall-deficient (L-form) Propionibacterium acnes which is dormant in cells of a host after initial infection (latent infection). As has also been reported, cardiac sarcoidosis is associated with enhanced expression of a type 1 helper T-cell cytokine (IL-1α, Il-2, IL-12p40, or INF-γ).

Studies have shown that cardiomyopathy cases (including dilated cardiomyopathy and hypertrophic cardiomyopathy) include many inflammatory cardiomyopathy cases which are triggered by viral infection (e.g., influenza infection), which are in the form of latent infection (rather than in a fulminant form (e.g., myocarditis)), and in which inflammation is prolonged by some abnormal autoimmune mechanism. That is, it has been shown that cardiomyopathy cases include inflammatory cardiomyopathy, in which infection-like inflammation is prolonged in the absence of a pathogen, as well as dilated cardiomyopathy, which is an end-stage form of inflammatory cardiomyopathy.

Such cardiomyopathy is a refractory severe disease which may result in clinical symptoms such as heart failure, myocardial conduction disorder, and fetal arrhythmia.

As shown in Table 24 below, in case 1 (cardiac sarcoidosis case), the quantitative value determined for TLR2 on a monocyte was found to be abnormally high.

Case 2: a patient with cardiomyopathy. In this case, infection symptoms and an increase in blood inflammatory marker level were not observed, but quantitative TLR2 value was found to fall within a range corresponding to infection. This suggests that case 2 may correspond to the aforementioned inflammatory cardiomyopathy.

It was found that the cause of infectious-agent-associated cardiomyopathy can be determined by using, as an index, the quantitative value determined for TLR2 on a monocyte. Thus, it was shown that quantitative TLR2 value can be used as an index for determining activation of systemic inflammation (including cardiac sarcoidosis) or myocardial inflammation (e.g., inflammatory cardiomyopathy).

TABLE 24

| | Blood | | | | | | |
|---|---|---|---|---|---|---|---|
| | collection | | | | | | |
| Case | date | Diagnosis | Age | Sex | TLR2 | WBC | CRP |
| 1 | 2006. 8. 3 | Cardiac | 57 | M | 13075 | 2700 | 0.07 |
| | | sarcoidosis | | | | | |
| 2 | 2006. 3. 7 | Hypertrophic | 72 | M | 6472 | 7690 | 0.11 |
| | | cardiomyopa- | | | | | |
| | | thy | | | | | |

### Example 18

### Atrial Fibrillation Arrhythmia

Atrial fibrillation arrhythmia is one of the most common arrhythmia cases and is roughly classified into two types: valvular atrial fibrillation and nonvalvular atrial fibrillation. Conceivably, in many valvular atrial fibrillation cases, arrhythmia results from disturbance of the conduction pathway from sinus node to atrioventricular node, which is caused by a load on atrial muscle or atrial enlargement, due to mitral stenosis or mitral insufficiency (valvular disease). In such a case, one of the causes of chronic valvular disease is considered previous childhood rheumatic fever (hemolytic streptococcus infection) (arteriosclerosis is also considered to be involved in chronic valvular disease). Meanwhile, nonvalvular atrial fibrillation is considered to be caused by inflammation in atrial muscle. Some theories suggest that viral infection of atrial muscle triggers nonvalvular atrial fibrillation. As has also been suggested, infection is involved in both types of atrial fibrillation arrhythmia.

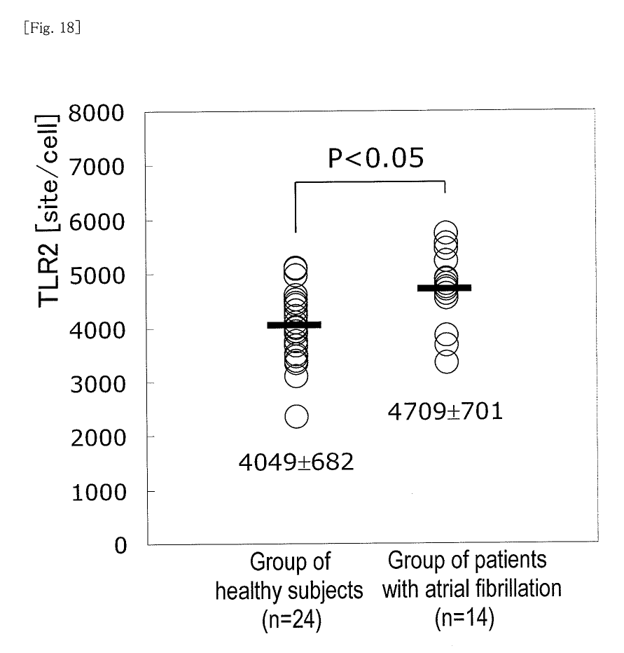

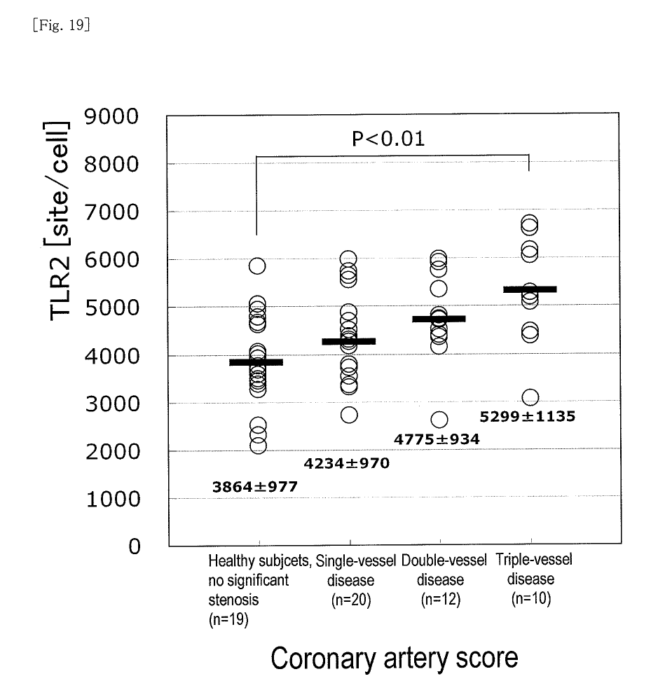

FIG. 18 shows comparison in quantitative value determined for TLR2 on a monocyte between a group of healthy subjects and a group of patients with atrial fibrillation arrhythmia (the mean age and male/female ratio of the former group are identical to those of the latter group). As shown in FIG. 18, the quantitative TLR2 value of the group of patients with atrial fibrillation is statistically significantly higher than that of the healthy subject group (but is not as high as that in the case of common infection). These data indicate that quantitative TLR2 value as determined by use of a blood sample (monocyte) from a patient with atrial fibrillation arrhythmia can be used as an index for understanding the degree of inflammation in the myocardium or valve of the patient, and quantitative determination of TLR2 count can provide useful information for arrhythmia treatment.

### Example 19

### Estimation of the Severity of Coronary Arteriosclerosis

Arteriosclerosis has been considered a pathological condition whose progression rate is complicatedly affected by numerous factors. Many studies have reported that arteriosclerosis is promoted by, among others, infection with bacteria such as chlamydia, cytomegalovirus, and periodontal bacteria. In this Example, the present inventors showed the relationship between severity of coronary arteriosclerosis and quantitative TLR2 value (FIG. 19). Test subjects were limited to patients with stable angina pectoris (i.e., angina pectoris patients who were considered not to be affected by cardiomyocyte necrosis due to myocardial ischemia). It was found that when the severity of coronary arteriosclerosis is determined by the number of coronary vessels with significant stenosis (i.e., when severity increases as the number of stenosed coronary arteries increases from one to two and to three), quantitative TLR2 value tends to increase in accordance with an increase in severity. In this clinical study, the severity of true arteriosclerosis (i.e., with no effect of myocardial necrosis) was evaluated by use of quantitative TLR2 value. Statistical data showed that a blood sample from a patient with coronary artery triple-vessel disease of high severity is highly likely to contain monocytes having high quantitative TLR2 value. Conversely, a high quantitative TLR2 value as obtained through quantitative determination may indicate the risk of progression or aggravation of arteriosclerosis. Thus, in view of many previous clinical findings in which progression of arteriosclerosis or ischemic event is inhibited by use of a drug for reducing such a risk (e.g., a lipid-lowering statin drug, a renin-angiotensin system inhibitor, or an angiotensin II receptor inhibitor), the present invention has a great clinical significance in that progression of arteriosclerosis can be monitored through quantitative determination of TLR2 count (i.e., a simple blood test).

1. A method of quantitative determination of sites, per cell of a test sample, at which an antibody is bound to an antigen protein (sites/cell), characterized by comprising:
preparing a calibration curve on the basis of fluorescent intensities obtained through measuring with a flow cytometer the amount of labeled antibodies against antigen protein which are bound to two or more groups of beads carrying known and different amounts of the antigen protein, and numeric values of the known amounts of the antigen protein, and
measuring, with the flow cytometer, labeled antibodies against antigen protein after they have been reacted with test cells derived from a blood sample of a test subject, whereby digitalization is effected through comparison and conversion between the calibration curve and a fluorescence intensity obtained.

2. A quantitative determination method according to claim 1, wherein the two or more groups of beads bearing known and different amounts of antigen protein are caused to coexist with test cells derived from a blood sample of a test subject, and the fluorescence-labeled antibody for antigen protein is allowed to react therewith, followed by measurement with a flow cytometer to obtain the following (1) or (2):
(1) a calibration curve prepared on the basis of fluorescence intensities of the beads versus the numerical values obtained for the antigen protein, and
(2) fluorescence intensity emitted from the test cells, wherein the above (1) and (2) are obtained in an assay system of a single flow cytometer.

3. A quantitative determination method according to claim 1, wherein the two or more groups of beads bearing known and different amounts of antigen protein are beads stored in a lyophilized state.

4. A quantitative determination method according to claim 1, wherein the antigen protein is a receptor protein present on the surface of a cell.

5. A quantitative determination method according to claim 1, wherein the antigen protein is a toll-like receptor protein.

6. A quantitative determination method according to claim 1, wherein the test cells are human leukocyte cells.

7. A quantitative determination method according to claim 6, wherein the test cells are human monocytes.

8. A quantitative determination kit for performing a quantitative determination method as recited in claim 1, the kit comprising, as components thereof, two or more groups of beads bearing known and different amounts of an antigen protein.

9. A quantitative determination kit according to claim 8, comprising, as components thereof, two or more groups of beads bearing known and different amounts of an antigen protein, and a labeled antibody against the antigen protein.
